(19) United States
(12) Patent Application Publication
(10) Pub. No.: US 2020/0221970 A1
(43) Pub. Date: Jul. 16, 2020

(54) DEVICE AND METHOD FOR PROCESSING AND VISUALIZING DATA RELATING TO CARDIAC AND PULMONARY CIRCULATION, OBTAINED BY MEANS OF AN ELECTRICAL IMPEDANCE TOMOGRAPHY DEVICE

(71) Applicant: Drägerwerk AG & Co. KGaA, Lubeck (DE)

(72) Inventors: Birgit STENDER, Lübeck (DE); Nicolas PILIA, Germersheim (DE)

(21) Appl. No.: 16/627,204

(22) PCT Filed: May 23, 2018
(86) PCT No.: PCT/EP2018/063440

§ 371 Date: Dec. 27, 2019

(30) Foreign Application Priority Data
Jun. 28, 2017 (DE) 10 2017 006 107.6

(51) Int. Cl. A61B 5/053; A61B 5/00; A61B 5/085; A61B 5/087

(52) U.S. Cl. CPC A61B 5/0536; A61B 5/004; A61B 5/085; A61B 5/7425; A61B 5/7246; A61B 5/7271; A61B 5/087

(57) ABSTRACT

A medical system (6000) includes an EIT module (30, 33, 8000), a ventilation module (7100), a dosing module (4), a data input module (50) and a control module (70). The control module (70) coordinates a breath-hold maneuver, which is carried out at the ventilation module (7100). The control module (70) coordinates a perfusion measurement and a data acquisition (50) of EIT data (3), which is carried out at the EIT module (30, 33, 8000). The control module (70) determines an indicator, which indicates a state of perfusion of the lungs, and makes this indicator of the state of perfusion of the lungs available.

### CROSS REFERENCE TO RELATED APPLICATIONS

This application is a United States National Phase Application of International Application PCT/EP2018/063440 filed May 23, 2018, and claims the benefit of priority under 35 U.S.C. §119 of German Application 10 2017 006 107.6, filed Jun. 28, 2017, the entire contents of which are incorporated herein by reference.

### TECHNICAL FIELD

The present invention pertains to a device and to a process/method for processing and visualizing data of an electrical impedance tomography device (EIT) in respect to a perfusion situation of the lungs, especially to a blood volume flow into the lungs and to a blood volume within the lungs.

### TECHNICAL BACKGROUND

Electrical impedance tomography devices (EIT) are known from the state of the art. These devices are configured and intended to generate an image, a plurality of images or a continuous image sequence by means of an array of electrodes by means of an image reconstruction algorithm from signals obtained by means of electrical impedance measurements and from data and data streams obtained therefrom. These images or image sequences show differences in the conductivity of different tissues of the body, especially of blood in the lungs and heart, as well as of breathing air in the lungs, as well as of the skeletal structure (costal arch, sternum, spine) surrounding the heart and the lungs in a horizontal plane. These images are useful for the assessment of states of the lungs in respect to perfusion and ventilation as well as in respect to the perfusion of the myocardium.

Thus, U.S. Pat. No. 6,236,886 describes an electrical impedance tomograph with an array of a plurality of electrodes, current feed at at least two electrodes and a process/method with an algorithm for image reconstructions to determine the distribution of conductivities of a body, such as bone, skin and blood vessels in a schematic configuration with components for signal acquisition (electrodes), signal processing (amplifiers, A/D converters), current feed (generator, voltage-current converter, current limitation) and components for controlling (μC). The electrical impedance tomograph makes it possible to visualize changes in conductivity within a cardiac cycle and the monitoring of blood flows in the heart and in the vessels.

It is explained in U.S. Pat. No. 5,807,251 that it is known in connection with the clinical application of EIT that a set of electrodes is provided, which are arranged at a defined distance from one another, for example, around the chest of a patient, in electrical contact with the skin, and an electrical current input signal or electrical voltage input signal is applied alternatingly between different pairs of electrodes or between all the possible pairs of electrodes arranged adjacent to one another. While the input signal is applied to one of the pairs of electrodes arranged adjacent to one another, the currents or voltages are measured between each mutually adjacent pair of the remaining electrodes and the measured data obtained are processed by means of an image reconstruction algorithm in order to obtain a visualization of the distribution of the specific electrical resistance over a cross section of the patient, around whom the electrode ring is arranged, and to display it on a screen.

The EIT is capable of distinguishing between ventilation and perfusion from the impedance differences between air/gas and blood in a spatially resolved manner. There are a plurality of heart beat cycles at the same time during one breath of a patient. Blood flows into the lungs and also out of the lungs with each heart beat.

The left ventricle of the heart or the left ventricle (ventriculus cordis sinister) or the left main chamber of the heart for receiving blood rich in oxygen from the pulmonary vein is usually called “left heart” or “region of the left heart” in medical terminology and likewise within the framework of the present application, usually in conjunction with the left atrium (atrium cordis sinistrum).

The right ventricle of the heart or the right ventricle (ventriculus cordis dexter) or the right main chamber of the heart, into which oxygen-depleted blood flows from the systemic circulation (vena cava), is called “right heart” or “region of the right heart” in medical terminology and likewise within the framework of the present application, usually in conjunction with the right atrium (atrium cordis dextrum).

The heart beat cycles display a certain variability in the heart beat rate and are asynchronous in relation to the breathing and are different from the respiration rate.

U.S. Pat. No. 9,384,549 B2 shows a device and a process/method for processing EIT data for improving the location-specific visualization of the perfusion of the lungs. An improved visualization of ventilation and perfusion is obtained in a common visualization due to a data processing with synchronization in time of ventilation-related and perfusion-related impedance changes.

Furthermore, data distribution of the EIT data into ventilation-related signals, cardiac and perfusion-related signals, as well as perfusion-related signals attributable to the cardiac activity and perfusion-related signals attributable to the lungs is described in U.S. Pat. No. 9,384,549 B2.

An interaction, as well as a combination of an electrical impedance tomography device (EIT) with a ventilator, is known from U.S. Pat. No. 7,162,296 B2. The ventilator is configured to initiate data and image acquisition at the EIT device. This makes possible, for example, a chronologically defined data and image acquisition at special times of ventilation, for example, to start a data and image acquisition during an inspiratory or expiratory pause, in order to find the slightest possible influence of the manner of ventilation or of the ventilation mode as an effect in the acquired EIT image.

Based on the EIT data and/or EIT images obtained in this manner and on information derived from these EIT data, different parameters or state variables can be determined in relation to the lungs of the patient in the EIT device, and they can be made available to the ventilator in order for the ventilator to be able to perform an adaptation of the ventilation on the basis of the EIT data and the different parameters or state variables. Such an adaptation of the ventilation is, for example, an adaptation of the positive end-expiratory pressure (PEEP), the ventilation rate (RR), and of the inhalation to exhalation ratio (I:E ratio).

A device for determining the regional distribution of an indicator for the lung perfusion is known from US 2015 0216443 A1. In addition to an impedance tomography unit with a plurality of electrodes arranged on the thorax and with a control and analysis unit, which is connected to the electrodes and is configured for feeding alternating current or alternating voltage in pairs, for acquiring voltage or current signals and for generating an EIT image in the plane of the electrodes arranged on the thorax, this device has a feed device for the intravenous feeding of a conductivity contrast medium.

Such a device is known, furthermore, from the article by Henning Liipschen et al.: “Determination of lung perfusion by means of electrical impedance tomography,” Biomedizinische Technik, 2010, 55, pp. 2-3. Such an EIT unit has a plurality of electrodes, which can be arranged on the thorax distributed over the circumference essentially in one plane. Further, a control and analysis unit is present, which is connected to the electrodes and which is set up consecutively to feed alternating current or alternating voltage to each pair of the plurality of electrodes and to receive the resulting voltage or current signals of the other electrodes as measured signals, and to reconstruct the impedance distribution in the plane from the measured signals. More precisely, what is determined here is not the impedance in absolute terms but its change in relation to a reference distribution. Such an EIT unit is described, for example, in EP 2 228 009 A1. Further, a manually actuated feed device (e.g., a syringe) is present in the prior-art device for the intravenous feed of a conductivity contrast medium.

Liquids, whose conductivity differs markedly from that of blood, may be used as conductivity contrast media. For example, hypertonic sodium chloride solutions with concentrations of up to 20% are commonly used.

Following the administration of a conductivity contrast medium, values of a conductivity dilution of regions of the lungs and heart can be recorded by means of the impedance tomography device (EIT) and represented, for example, in the form of conductivity dilution curves, as they are shown in DE 10 2012 214 786 A1.

Impedance changes develop during the inflow of contrast medium following the administration of a conductivity contrast medium, first in the region of the right heart in as well as above and below the array of EIT electrodes (plane), of the thorax in the region of the right heart, after which the contrast medium then leaves the region of the right heart in the direction of the lungs and an impedance change will develop in the plane of the thorax in the region of the lungs, after which the contrast medium then flows back into the region of the left heart, and an impedance change will then develop in the plane of the thorax in the region of the left heart.

A qualitative assessment of the perfusion situation of the lungs is possible by means of one or more conductivity dilution curves, because the status of the function of the blood circulation from the heart into the lungs and back to the heart can be determined by means of the impedance tomography device (EIT) on the basis of the detected impedance changes. Further qualitative determinations of the perfusion situation of the lungs, especially of the distribution of the blood volume flow (pulmonary blood flow, PBF) in the lungs and of the blood volume (pulmonary blood volume, PBV) within the lungs are not possible directly on the basis of the regional distribution of the conductivities, but only by a subsequent analysis of a plurality of conductivity dilution curves. Suitable methods for estimating regional perfusion situations in the lungs are described in the scientific literature, for example, in an article by Borges, J. B., Suarze-Sipmann, F., Bohm, S. H., Tusman, G., Melo, A., Maripuu, E., Sandstrom, M., Park, M., Costa, E. L., Hedenstierna, G., and Amato, M.: “Regional lung perfusion estimated by electrical impedance tomography in a piglet model of lung collapse,” Journal of Applied Physiology, 112 (1), January 2012, pp. 226-228.

### SUMMARY

An object of the present invention is to provide a device and a process/method as well as a system for processing and visualizing data obtained by means of an electrical impedance tomography device, which provides a quantifiable analysis in respect to a perfusion situation of the lungs.

Another object, which is closely linked with this object, arises from the task of achieving an improvement of the possibility of a regional analysis in respect to the perfusion of regions of the lungs and of regions of the heart on the basis of the data provided by an electrical impedance tomography device or by means of an improved electrical impedance tomography system.

Features and details that are described in connection with the process/method according to the present invention for processing and visualizing data of an electrical impedance device (EIT) in respect to a perfusion situation of the lungs are, of course, also applicable in connection with and in regard to the device suitable for carrying out the process/method or the system and also vice versa, so that reference is and can always mutually be made to the individual aspects of the present invention concerning the disclosure.

Furthermore, the process/method may also be provided as a computer program or as a computer program product, so that the scope of protection of the present application also extends to the computer program product and to the computer program.

According to a first aspect of the present invention, data obtained in a process/method according to the present invention by means of an electrical impedance tomography device are processed in a sequence of steps, so that a quantifiable analysis in respect to a perfusion situation of the lungs is made possible. The process/method according to the present invention for processing and visualizing data obtained by means of an electrical impedance tomography device (EIT) in respect to the perfusion of the heart and lungs of a patient is divided into a sequence of steps comprising the following steps:
provision of a data set of pixels (a data set of pixels values)with impedance signals (impedance signal values), which represent a superimposition of cardiac-related signal components in regions of the lungs, of the heart or of the thorax with signal components, which represent a spread of a predefined quantity of liquid of an indicator solution in regions of the lungs, of the heart or of the thorax during a breath-hold phase, on the basis of the data obtained by means of the electrical impedance tomography device (EIT) over a signal waveform located within an observation/analysis period,
provision of a data set, which represents information in respect to at least one cardiac function, especially a heart rate,
determination of a data set containing cardiac-related impedance changes (CRIC) with information that indicates a pulsatile cardiac activity, especially a heart beat rate or a pulse beat of the heart in regions of the lungs, of the heart or of the thorax, on the basis of the data set of pixels and on the basis of the data set containing information in respect to the at least one cardiac function, especially on the basis of the heart beat rate or of the pulse beat of the heart,
determination of a data set, which indicates a relative distribution of a signal power or power density or a relative amplitude distribution of the cardiac-related impedance signals in a predefined frequency range, on the basis of a data set containing cardiac-related impedance changes (CRIC) with pieces of information that indicate the pulsatile cardiac activity,
determination of a data set, which indicates a time information or a phase information of the cardiac activity in regions of the lungs, of the heart or of the thorax, on the basis of the data set containing cardiac-related impedance changes (CRIC) with pieces of information that indicate the pulsatile cardiac activity, especially a heart beat rate or a pulse beat of the heart in regions of the lungs, of the heart or of the thorax,
determination of two location-specific data sets classified according to an evaluation criterion on the basis of the data set that indicates the relative distribution of power or power density or the amplitude distribution of the cardiac-related impedance signals and/or on the basis of the data set containing pieces of time or phase information, which indicates the cardiac activity in regions of the lungs, of the heart or of the thorax, wherein a data set of the two location-specific data sets indicates a subset in the data set of pixels with impedance signals, in which subset a blood volume flow is directed from the lungs to the heart, and an additional data set of the two location-specific data in the data set of pixels with impedance signals, in which subset a blood volume flow is directed from the heart to the lungs,
determination and provision of an indicator, which indicates a state of perfusion of the lungs on the basis of the two location-specific data sets and on the basis of the data set of pixels with impedance signals, and
determination and provision of a first control signal, which indicates the indicator indicating the state of perfusion of the lungs.

The provision of the data set of pixels with impedance signals may be configured as provision of a data set of EIT data in a different form. EIT data are defined in the sense of the present invention as the following signals or data:
EIT raw data, i.e., measured signals acquired with an EIT device by means of a group of electrodes or by means of an electrode belt, such as voltages or currents, associated with electrodes or with groups of electrodes or with positions of electrodes or with groups of electrodes on the electrode belt.
EIT image data, i.e., data or signals that were determined with a reconstruction algorithm from the EIT raw data and represent local impedances, impedance differences or impedance changes of regions of the lungs or regions of the lungs and of the heart of a patient.
classified EIT data, i.e., EIT image data or signals, which are presorted or preclassified according to predefined criteria. The classification may be implemented, for example, as a standardized distribution into EIT data or signals, which represent cardiac- and perfusion-related impedances (cardiac and perfusion-related signals), impedance differences or impedance changes, and into EIT data or signals, which represent ventilation-related impedances, impedance differences or impedance changes (ventilation-related signals).
Specially classified EIT data, i.e., EIT image data or signals, which are presorted or preclassified according to special predefined criteria. Such a special classification may be implemented, for example, as a distribution into EIT data or signals, which comprise essentially perfusion-related impedances (perfusion-related signals), impedance differences or impedance changes of the lungs, and into EIT data or signals, which comprise perfusion-related impedances (cardiac-related signals), impedance differences or impedance changes of the heart, which are caused or elicited by changes in the blood volume in regions of the heart and of the major blood vessels thereof.

The EIT data may have been generated under special conditions of signal acquisition. Special conditions of signal acquisition arise, for example, from the boundary conditions of breathing and ventilation in connection with the feeding (dispensing) and administration of the predefined quantity of liquid of the indicator solution into the blood circulation.

Predefined quantities of liquid of an indicator solution or doses of an indicator solution are called a bolus or a bolus quantity in clinical usage.

Suitable locations on the patient's body for feeding and administering the predefined quantity of liquid of the indicator solution into the blood circulation are venous blood vessels. The administration is performed either intravenously, for example, via a central venous catheter, or via the proximal lumen of a Swan-Ganz catheter, or the administration is performed peripherally, for example, via the veins of the arm.

Suitable indicator solutions have a conductivity contrast against the blood. Suitable indicator solutions are, for example, sodium chloride solutions. At a concentration different from 0.9%, the osmotic concentration (osmolarity) of this contrast medium is different from that of the blood, and sodium chloride solutions should therefore be used carefully. Therefore, the quantity administered, the number of repetitions for an averaging and the concentration should therefore be selected to be as low as possible in case of a measurement with this indicator.

The EIT data may be limited to the defined observation/analysis period. or they may have been obtained as a subset of a data set of impedance values, which data set was acquired over a longer period, or of values or data derived from impedance values. The observation/analysis period may be obtained in connections with breathing and/or ventilation and/or in connection with the feeding and administration of the predefined quantity of liquid of the indicator solution into the blood circulation.

Especially ventilation modes, breath-hold maneuvers for generating one or more breath-hold phases, are obtained as observation/analysis periods in connection with the boundary conditions of breathing and ventilation, especially in case of a coordinated operation of an EIT device and a ventilator.

Based on a time course of relative impedance changes Z(t) as a reference time curve B(t), wherein the reference time curve B(t), often also called a so-called “baseline,” the following variants are obtained as the observation/analysis periods in connection with the feed and administration of the predefined quantity of liquid of the indicator solution, beginning with the breath-hold maneuver and the administration of the indicator solution (bolus), as an essentially exponential drop B(t) in the time curve of the relative impedance change Z(t):

Variant A: A time interval from the beginning to the end of a dynamic change of a mean or added-up impedance over a plurality of pixels in the EIT images.

The time interval (tₛₜₐᵣₜ, t_{end}) of the dynamic change, caused by the administration of the indicator solution (bolus) in a time series of relative impedance values, which characterize the relative impendence time curve, is now averaged or added up and defined by the start time tₛₜₐᵣₜ and the end point t_{end}, wherein the start time tₛₜₐᵣₜ can be determined according to Formula 1 or Formula 2.

$$\begin{matrix}{t}_{start} = min \{ t \vert \vert Δ Z ( t ) \vert > {c}_{s 1} ⋀ {\int }_{t \cdot Δ \text{?}}^{t} \vert Δ Z ( t ) \vert dt > {c}_{s 2} ⋀ \frac{d}{dt} \vert Δ Z ( t ) \vert > {c}_{s 3} \} , & Formula 1 \\ {t}_{end} = min \{ t \vert t > {t}_{start} ⋀ \vert Δ Z ( t ) \vert < {c}_{e 1 } ⋀ {\int }_{t \cdot Δ \text{?}}^{t} \vert Δ Z ( t ) \vert dt < {c}_{e 2} \} , \\ \text{?} \text{indicates text missing or illegible when filed} & Formula 2\end{matrix}$$

The start time tₛₜₐᵣₜ is characterized by an absolute deviation |ΔZ(t)|=|Z(t)−B(t)| in the relative impedance time curve Z(t) from the reference time curve B(t) (baseline), which exceeds a defined constant value cₛ₁ in combination with an integration over the time interval of the duration ΔTs as well as with a change over time in this absolute deviation, which exceed defined constant values cₛ₂ and cₛ₃.

The end time t_{end} is characterized by the approximation of the relative impedance curve Z(t) to the reference time curve B(t), characterized by an absolute deviation |ΔZ(t)| that is essentially constant over time below a constant value cₑ₁ in combination with a value for the integral over the time interval of the duration ΔTₑ below a constant value cₑ₂.

The state at the end point after the administration of the indicator solution (bolus) is a state of equilibrium, which is characterized by the conclusion of all dynamic processes and equalization processes in the cardiovascular system.

Variant B: A time interval |t_{rH,start}, t_{lH,end}| from the beginning to the end of an absolute deviation in the impedance time curve Z_{rH}(t) against the reference time curve B_{rH}(t) (baseline).

The time interval |t_{rH,start},t_{lH,end}| between a start time t_{rH,start} according to Formula 3 of an absolute deviation |ΔZ_{rH}(t)|=|Z_{rH}(t)−B_{rH}(t)| in the relative impedance time curve Z_{rH}(t) against the reference time curve B_{rH}(t) (baseline), which is caused by the administration of the indicator solution (bolus), is detected in an image area that represents regions of the right heart, and an end time t_{lH,end} following in the time curve according to Formula 4 and analogously to Formula 3 of an absolute deviation |ΔZ_{lH}(t)|=|Z_{rH}(t)−B_{rH}(t)| caused by the administration of the indicator solution (bolus) in the relative impedance time curve Z_{lH}(t) against the reference time. The curve B_{lH}(t) (baseline) is detected in an image area that represents regions of the left heart.

$$\begin{matrix}{t}_{rH , start} = min \{ t \vert \vert Δ {Z}_{rH} ( t ) \vert > {c}_{rH , s 1} ⋀ {\int }_{t \cdot Δ {t}_{rH , s}}^{t} \vert Δ {Z}_{rH} ( t ) \vert dt > {c}_{rH , s 2} ⋀ \frac{d}{dt} \vert Δ {Z}_{rH} ( t ) \vert > {c}_{rH , s 3} \} , & Formula 3 \\ {t}_{\text{?} H , start} = min \{ t \vert t > {t}_{rH , start} ⋀ \vert Δ {Z}_{\text{?}} ( t ) \vert < {c}_{\text{?} , e 1} ⋀ {\int }_{t \cdot Δ {T}_{\text{?}}} \vert Δ {Z}_{\text{?}} ( t ) \vert > {c}_{\text{?} , e 2} \} , \\ \text{?} \text{indicates text missing or illegible when filed} & Formula 4\end{matrix}$$

Variant C: In case of a coordinated operation of the EIT with the device for administering the infusion, configured, for example, as a manual infusion, manual or automatic syringe or peristaltic pump, or as a so-called power injector, a predetermined time interval, which can be defined as a predefined time interval beginning from a manual or automated dispensing of the indicator solution (bolus) with a dispensing period of a few seconds, for example, two to five seconds, to the end of a set time interval, for example, 20 to 50 seconds, or, as an alternative, until the detection of an end time t_{end} or t_{1H,end}, calculated corresponding to Formula 2 or Formula 4, is obtained as a variant of a duration of an observation/analysis period.

Coordinated operation of an EIT device and a ventilator, as it is described in DE 103 01 202 B3, makes it possible, for example, to facilitate the configuration of analysis periods without breathing activities of the patient or the operation of the ventilation by the ventilator being able to effectively influence data of the data set of pixels with impedance signals, which represent a superimposition of cardiac-related signal components in regions of the lungs, of the heart or of the thorax with signal components that represents the spread of a predefined quantity of liquid of an indicator solution in regions of the lungs, of the heart or of the thorax. A breath-hold maneuver may be configured during the phase of inhalation (inspiratory hold maneuver) or the phases of exhalation (expiratory hold maneuver) or even as a change in a pressure-controlled ventilation mode to a ventilation mode with a constant pressure level (CPAP). Possible configurations and applications of breath-hold maneuvers in electrical impedance tomography are described, for example, in WO 2009 035 965 Al.

A breath-hold maneuver can be embodied, for example, in the following manner as a coordination between an EIT device and a ventilator:
Step 1: Preparations are made for a breath-hold maneuver by the user on a ventilator,
Step 2: a perfusion measurement is started on the EIT device,
Step 3: the EIT device sends a request for starting a breath-hold maneuver to the ventilator, and
Step 4: the ventilator initiates the breath-hold maneuver and sends a confirmation on the success of the start of the maneuver to the EIT device.

The indicator solution is subsequently administered (injection) either directly thereafter or after a fixed, short waiting period or after a stable reference time curve Bzi . . . n(t) (stable baseline) has been detected in an impedance time curve ΔZi . . . n(t), which is characteristic of a larger number of pixels in the EIT image. As an alternative, the injection may also be started in an automated manner.

Such or further automation is made possible especially by the inclusion of an actuatable infusion source. For example, EIT data and image acquisition with synchronization of the administration of the indicator solution (bolus) with a breath-hold maneuver is made possible by means of an inclusion of a syringe pump in the coordinated operation of the EIT device and ventilator, so that the provided data set of pixels with impedance signals, which represents a superimposition of cardiac-related signal components in regions of the lungs, of the heart or of the thorax with signal components, which represent the spread of a predefined quantity of liquid of an indicator solution in regions of the lungs, of the heart or of the thorax, are adapted to a suitable analysis period on the basis of the data obtained by means of the electrical impedance tomography device (EIT) on a signal waveform located within an observation/analysis period by the manner of coordination of the EIT device, syringe pump and ventilator, without sorting or postprocessing of the data concerning the position of the analysis period in the observation/analysis period being necessary.

The data set, which represents pieces of information in respect to at least one cardiac function, especially a heart rate, may be provided here from different information sources. Information on the heart rate or the pulse beat may be provided by different devices or device constellations, which are configured to detect a pulsatile measured value, for example, by
a device with functions of an electrocardiogram (EKG), e.g., configured as a physiological monitor,
a device with functions for measuring or determining an oxygen saturation or an oxygen partial pressure measurement by means of photoplethysmography (SpO₂), configured, e.g., as a physiological monitor or a device for measuring the oxygen partial pressure,
an EIT device with integrated EKG functionalities, and
an EIT device with integrated functionalities for measuring or determining an oxygen saturation or oxygen partial pressure measurement by means of photoplethysmography (SpO2).

The data set, which represents information concerning at least one cardiac function, especially a heart rate, may be provided here, for example, by means of an electrical or optical serial (RS232, RS485, USB, IRDA) or parallel (IEEE488) data interface or even by means of telemetric data transmission (GSM, UMTS, Bluetooth) to the EIT device.

The data set, which represents information concerning at least one cardiac function, especially a heart rate, may, however, also be provided, as an alternative, by a data coordination in a data network (network, server network, Intranet, Internet, Cloud) via different components (servers, routers, switches, hubs) of a data network (LAN, WLAN), for example, in the form of a Patient Area Network (PAN) with optional connection to a Patient Data Management System (PDMS) in the hospital or in a network of a plurality of hospitals in a wired, wireless or optical (glass fiber network) manner. Such a “Patient Area Network (PAN” is described in US 2008/000479 A1.

A cloud or cloud computing is defined in the technical usage of information and network technology as the execution of programs, e.g., computation routines, processing of data or measured signals by means of control and regulation algorithms, data processing, data coordination (database, data set management) or similar, wherein these programs or subroutines of the programs are not installed on local computer units or devices, but are polled from a distance on another computer or distributed among a plurality of other computers, for example, in a hospital network (Intranet) or in a worldwide network system (WWW, Internet).

The determination of the data set containing cardiac-related impedance changes (CRIC) with pieces of information that indicate a pulsatile cardiac activity, especially a heart beat rate or a pulse beat of the heart in regions of the lungs, of the heart or of the thorax, on the basis of the data set of pixels and on the basis of the data set containing pieces of information concerning the at least one cardiac function, especially on the basis of the heart beat rate or of the pulse beat of the heart, can be carried out in different manners. The heart rate can be determined as follows on the basis of the data set of pixels: In a first variant, a signal, which is representative of a mean value or of a mean value of all elements or of a subset of the data set, from the data set containing cardiac-related impedance changes (CRIC) or from a suitable subset.

In a second variant, a power density spectrum is calculated from the data set of pixels with impedance signals, and the heart rate is determined from the power spectrum in a characteristic frequency range, preferably by means of a robust process/method.

A characteristic frequency range in a physiologically relevant range is, for example, a frequency range above a characteristic frequency of 0.67 Hz for an adult, which corresponds to a heart beat rate of 40 beats per minute.

A characteristic frequency range in a physiologically relevant range is, for example, a frequency range above a characteristic frequency of 2 Hz for a child at an age of 2 years, which corresponds to a heart beat rate of 120 beats per minute. A robust process/method is, for example, a parametric approach for estimation by means of an autoregressive model, as it is described, for example, in a scientific paper by Takalo R.; Hytti H.; Ihalainen H.: “Tutorial on Univariate Autoregressive Spectral Analysis,” Journal of Clinical Monitoring and Computing, 2005, 19: pp. 402-404. The manner of signal processing, especially the selection of the spectral analysis or transmission/blocked ranges of filters, can be derived here from the data set containing pieces of information concerning the at least one cardiac function, especially on the basis of the heart beat rate or of the pulse beat of the heart, because typical respiration rates are lower than typical heart rates by a factor of four to five.

The determination of a data set, which indicates a relative distribution of a signal power or power density or a relative amplitude distribution of the cardiac-related impedance signals in a predefined frequency range, on the basis of the data set of pixels and on the basis of the data set containing pieces of information concerning the at least one cardiac function, especially on the basis of the heart beat rate or of the pulse beat of the heart, is carried out here as will be described below.

An amplitude spectrum or an amplitude density spectrum is calculated for each element of the data set of pixels, for example, by the application of a Fast Fourier Transformation (FFT). Amplitude values in a range around the typical and known heart rate are added up or are added up and then averaged in each of the amplitude spectra.

As an alternative, it is also possible to use as the basis a power spectrum or a power density spectrum to determine the data set that indicates a relative distribution of a signal power or power density or a relative amplitude distribution of the cardiac-related impedance signals in a predefined frequency range, and the values of the data set are added up or averaged in a range around the typical and known heart rate.

The determination of a data set that indicates time information or phase information of the cardiac activity in regions of the lungs, of the heart or of the thorax, on the basis of the data set of pixels and on the basis of the data set containing information concerning the at least one cardiac function, especially on the basis of the heart beat rate or of the pulse beat of the heart, is carried out here on the basis of a phase spectrum.

The time or phase information of the cardiac activity in regions of the lungs, of the heart or of the thorax can be determined on the basis of the phase spectrum, which was likewise calculated, for example, as was already described before in connection with the amplitude spectrum, by the application of an FFT. These values of the phase spectrum are averaged in a range around the typical and known heart rate analogously to the amplitude spectrum. As an alternative, a similarity indicator may be determined in pairs between all elements or a plurality of elements of the data set containing cardiac-related impedance changes (CRIC); for example, it is possible to determine a similarity indicator that can be determined by means of the determination of a linear correlation coefficient on the basis of elements of the data set containing cardiac-related impedance changes (CRIC).

The determination of two location-specific data sets classified according to an evaluation criterion on the basis of the data set that indicates the relative distribution of power or power density or the amplitude distribution of the cardiac-related impedance signals and/or on the basis of the data set containing time or phase information, which indicates the cardiac activity in regions of the lungs, of the heart or of the thorax, is carried out such that an approach in which a set of elements in the image, i.e., within the pixels of the original data set, forms a contiguous area, is selected as the basis for the evaluation criterion. This basis is combined with additional criteria, so that different configuration variants of the evaluation criterion are obtained, as they will be described below. A respective entry from the data set, which indicates the relative distribution of power or power density or the amplitude distribution of the cardiac-related impedance signals, and/or from the data set containing time- or phase-specific information or the similarity indicator, is assigned to each element of the original data set of pixels. A shorter notation as “data set of the relative amplitude or power distribution” or “data set of the relative amplitude or similarity indicator,” “data set containing similarity indicators” or “data set containing time or phase information” will also be used in the further course of the description for the data set that indicates the relative distribution of power or power density or the amplitude distribution of the cardiac-related impedance signals and/or for the data set containing time or phase information or the similarity indicator.

The determination of the location-specific data set, in which a blood volume flow is directed from the right heart to the lungs, can be determined as the set of the elements that form a contiguous area in the image, i.e., within the pixels of the original data set and whose entries in the data set of the relative amplitude and power distribution exceed a defined percentage of a predefined maximum, for example, a percentage of 40% or 50% of the maximum of the relative amplitude or power distribution.

One of the following additional criteria is necessary for this as an additional criterion for determining the location-specific data set, in which criterion a blood volume flow is directed from the right heart to the lungs:
An area, in which the sum of the entries in the data set of the relative amplitude or power distribution is obtained as a maximum, is selected among all the contiguous areas in the image,
an area, which contains most of the image elements, is selected among all the contiguous areas in the image, and
an area, i.e., for example, an area, for which a certain probability is obtained on the basis of studies, investigations and analyses of a large number of patient data, is selected among all the contiguous areas in the image on the basis of additional pieces of information.

The determination of the location-specific data set, in which data set a blood volume flow is directed from the right heart to the lungs, can be determined in a first alternative variant as the set of the elements that form a contiguous area in the image, i.e., within the pixels of the original data set, and whose entries in the data set of the relative amplitude or power distribution exceed a certain percentage of a predefined maximum, for example, a percentage of 5% or 10% of the maximum of the relative amplitude or power distribution, and whose entries in the data set containing time or phase information or similarity indicator are in a defined range and in whose entries the similarity indicator for at least one other element of the contiguous area exceeds a predefined value. Exceeding is present in respect to such a similarity indicator, for example, in case of a linear correlation coefficient of about >0.80 or >0.85. One of the following additional criteria is necessary for this as an additional criterion for determining the location-specific data set, in which criterion a blood volume flow is directed from the right heart to the lungs:
An area, in which the sum of the entries in the data set of the relative amplitude or power distribution has a maximum, is selected among all the contiguous areas in the image,
an area, which contains most of the image elements, is selected among all the contiguous areas in the image, and
an area is selected among all the contiguous areas in the image on the basis of additional pieces of information, i.e., for example, an area is selected, for which a certain probability is obtained based on studies, investigations and analyses of a large number of patient data.

The determination of the location-specific data set, in which data set a blood volume flow is directed from the right heart to the lungs, can be determined in a second alternative variant as the set of the elements that form a contiguous area in the image, i.e., within the pixels of the original data set, and whose entries in the data set of the relative amplitude or power distribution exceed a certain percentage of a predefined maximum, for example, a percentage of 5% or 10% of the maximum of the relative amplitude or power distribution, and whose entries in the data set containing time or phase information are related to entries of signals, which indicate a cardiac activity, for example, periods in EKG data with significant signal elements, e.g., the so-called “R-wave” or the so-called “QRS complex.”

The determination of the location-specific data set, in which data set a blood volume flow is directed from the lungs into the region of the left heart, is determined as the set of the elements that form a contiguous area in the image, i.e., within the pixels of the original data set, and whose entries in the data set of the relative amplitude or power distribution exceed a certain percentage of a predefined maximum, for example, a percentage of 5% or 10% of the maximum of the relative amplitude or power distribution. One of the following additional criteria is necessary for this as an additional criterion for determining the location-specific data set, in which data set a blood volume flow is directed from the lungs to the heart:
the entries in the data set containing time or phase information have phase positions from 0.45⋅THeart to 0.55⋅THeart in relation to the duration of the period of the cardiac activity THeart and are thus obtained in relation to elements of the data set in which a blood volume flow is directed from the heart to the lungs, and, as an alternative, this phase position can be determined, for example, on the basis of a phase shift relative to the so-called “R-wave” or the so-called “QRS complex” in EKG data, wherein the duration (preejection time) between excitation and the start of the contraction of the main chamber is also taken into account in addition to the relation to the duration of the period,
for at least one element of the data set containing similarity indicators, the similarity indicator of these entries is given in relation to elements of the data set, in which a blood volume flow is directed from the heart to the lungs, such that an especially weak similarity is given, for example, i.e., there is a linear correlation coefficient of <0.7, and
an area, i.e., for example, an area, for which a certain probability is obtained on the basis of studies, investigations and analyses of a large number of patient data, is selected among all the contiguous areas in the image.

The determination of the indicator, which indicates a state of perfusion of the lungs, and the determination and the provision of the first control signal, which indicates the indicator indicating the state of perfusion of the lungs, are carried out on the basis of the determined two location-specific data sets and on the basis of the data set of pixels containing impedance signals. The determined two location-specific data sets represent a region of the heart (Region of Interest, ROI A), in which a blood volume flow is directed from the lungs to the heart, and a region of the heart (Region of Interest, ROI B), in which a blood volume flow is directed from the heart to the lungs. These two regions (ROI A, ROI B) thus represent the so-called pulmonary circulation, i.e., the circulation in which oxygen-depleted blood rich in carbon dioxide is delivered from the heart to the lungs and blood with high oxygen level and low carbon dioxide level is delivered back to the heart following the CO₂/O₂ gas exchange in the lungs and is delivered from there into the body into the so-called systemic circulation for supplying organs and muscles with oxygen.

The first control signal may be used for output to a display unit connected directly or indirectly to the EIT device, for transmission into a data network system, data network (LAN, WLAN, PAN, Cloud).

The functionalities described above in the steps with the provision of a data set of pixels with impedance signals, which represent a superimposition of the cardiac-related signal components with signal components that represent a spread of the predefined quantity of liquid of the indicator solution, the determination of the data set containing cardiac-related impedance changes (CRIC), the determination of the data set that indicates a relative distribution of a signal power or a relative amplitude distribution of cardiac-related impedance signals in a predefined frequency range, and the determination of the date set that indicates time or phase information of the cardiac activity may be carried out on a computer. It is likewise possible, as was explained before in connection with the provision of the data set that contains the pieces of information concerning at least one cardiac function, especially a heart rate, that these functionalities or parts of these functionalities, as well as the steps and functionalities described in the further embodiments of the process/method, are carried out in a data network (network, server network, Intranet, Internet, Cloud, Cloud Computing) via different components (servers, routers, switches, hubs) of a data network (LAN, WLAN), for example, in the form of a Patient Area Network (PAN) with optional connection to a Patient Data Management System (PDMS) in the hospital or in a network of a plurality of hospitals in a wired, wireless or optical (glass fiber network) manner in a form of the Cloud Computing described in more detail before.

The two location-specific data sets or the two location-specific and flow- and perfusion-specific data sets are processed in the following manner for a more refined determination of the indicator, which indicates a state of perfusion of the lungs, wherein a determination of a blood volume flow (PBF) through the lungs and/or a determination of a blood volume (PBV) within the lungs are advantageous as exemplary embodiments of refined determination.

An especially robust approach to the more refined determination of the indicator, which indicates a state of perfusion of the lungs, is based, for example, on an estimation of the so-called residual function by means of deconvolution. Deconvolution designates the reversal of the folding operation (convolution). Deconvolution is used in image processing, for example, to sharpen images. Since images of a ventilation situation or perfusion situation of the lungs, which images are determined from impedance values following the application of the image reconstruction, are likewise obtained in electrical impedance tomography, image processing routines based on deconvolution can also be applied to these EIT images and, especially within the framework of the present invention, also to a series of EIT images, which show or represent a passage (dilution) of the administered indicator solution through the tissues in the thorax, i.e., through lung regions and cardiac regions. The underlying indicator dilution theory was already described in connection with cerebral perfusion imaging based on time series of radiological methods with contrast medium administration. Especially Paul Meier and Kenneth L. Zierler: “On the theory of the indicator dilution method for measurement of blood flow and volume,” Journal of Applied Physiology, 1954, 6(12): pp. 733-743, Yoshiharu Ohno, Hiroto Hatabu, Kenya Murase, Takanori Higashino, Hideaki Kawamitsu, Hirokazu Watanabe, Daisuke Takenaka, Masahiko Fujii, and Kazuro Sugimura: “Quantitative assessment of regional pulmonary perfusion in the entire lung using three-dimensional ultrafast dynamic contrast-enhanced magnetic resonance imaging: Preliminary experience in 40 subjects,” Journal of Magnetic Resonance Imaging, 2004, 20(3): pp. 356-357, and Leif Ostergaard, Robert M. Weisskopf, David A. Chesler, Carsten Gyldensted, Bruce R. Rosen: “High resolution measurements of cerebral blood flow using intravascular trace bolus passages. Part I: Mathematical approach and statistical analysis,” Magnetic Resonance in Medicine, 1996, 36(5): pp. 715-718 shall be mentioned as publications in this connection.

If, for example, one location-specific and flow- and perfusion-specific data set represents the impedance time curve in a feeding vessel, this data set can be considered to be an input signal of a dynamic system, often also called a so-called “arterial input function” (AIF). The dynamic system characteristic is described by the residue function, which characterizes at the same time the percentage of indicator solution still remaining in the tissue. The course of an impedance change ZROI(t) in a region being considered (region of interest, ROI) as a response to the course of the arterial input function can consequently be considered to be a folding of AIF Z_{AIF}(t) with the residue function ROR(t):

Z_{ROI}(t)=Z_{AIF}(t)⊗[R₀R(t)] Formula 5

After estimating the residue function by means of deconvolution, the pulmonary blood volume flow (PBF) and the pulmonary blood volume (PBV) in the particular region are obtained as follows:

$$\begin{matrix}{PBF}_{ROI} = {R}_{0} & Formula 6 \\ {PBV}_{ROI} = \frac{{\int }_{- ∞}^{∞} {Z}_{ROI} ( t ) dt}{{\int }_{- ∞}^{∞} {Z}_{AIF} ( t ) dt} & Formula 7\end{matrix}$$

Methods for estimating blood flows are described, for example, in the scientific treatise of Leif Ostergaard, Robert M. Weisskopf, David A. Chesler, Carsten Gyldensted, Bruce R. Rosen: “High resolution measurements of cerebral blood flow using intravascular trace bolus passages. Part I: Mathematical approach and statistical analysis,” Magnetic Resonance in Medicine, 1996, 36(5): pp. 715-718.

A less robust approach for the determination of relative perfusion distributions, which approach does, however, require less computing, is the so-called “Maximal Slope Method,” which is described, for example, in Miles K A: “Measurement of tissue perfusion by dynamic computed tomography,” British Journal of Radiology, 1991, 64: pp. 409-410, and Konstas A A, Goldmakher G V, Lee T Y, Lev M H: “Theoretic basis and technical implementations of CT perfusion in acute ischemic stroke, Part 1: Theoretic basis,” American Journal of Neuro Radiology, 2009, 30: pp. 663-667.

$${PBF}_{ROI} = \frac{max ( \frac{{dZ}_{ROI} ( t )}{dt} )}{max ( {Z}_{AIF} ( t ) )} \qquad Formula 8$$

However, perfusion distributions have been determined so far with this approach on the basis of EIT data only without the determination of an arterial input function (AIF) via the time derivation as relative perfusion distributions, as is described, for example, in Borges, J. B., Suarze-Sipmann, F., Böhm S. H., Tusman, G., Melo, A., Maripuu, E., Sandström, M., Park, M., Costa, E. L., Hedenstierna, G., Amato, M.: “Regional lung perfusion estimated by electrical impedance tomography in a piglet model of lung collapse,” Journal of Applied Physiology, 112(1), January 2012, pp. 226-228.

The mean transit time (MTT) is another parameter, which is commonly used to characterize a state of perfusion:

$${MTT}_{ROI} = \frac{{PBV}_{ROI}}{{PBF}_{ROI}} \qquad Formula 9$$

A time Tₘₐₓ, at which the residue function (Formula 5) reaches its maximum, is likewise a commonly used perfusion parameter.

If the selected region (ROI) characterizes a region in the left heart, the parameters do not characterize the state of perfusion of a region in the lungs, but the state of the entire lung.

In an alternative embodiment, the above-mentioned calculations of the indicator, which describes a state of perfusion of the lungs (PBF, FBV and MTT) in case of calculation via the aforementioned residue function, can also be carried out, on the basis of a comparison of the amplitudes of the cardiac-related impedance changes with the relative impedance changes, which are due to the administration of the indicator, on the basis of the entire signal component in the respective selected regions (ROI), i.e., not only on the basis of the flow- and perfusion-specific component caused by the indicator solution.

A period, which characterizes the residence time of the indicator solution in the pulmonary circulation, is preferably used, for example, as an analysis period within the observation/analysis period. The beginning of this period is characterized by the initial detection of a change in the flow- and perfusion-specific component of the EIT data, which is explained in connection with the variants A, B, C of observation/analysis periods in connection with the feed and administration of the predefined quantity of liquid of the indicator solution, for example, as a significant global impedance change or as a local significant impedance change in the region of the right heart at the beginning of the inflow of the indicator solution.

A time, at which the indicator solution has again left the pulmonary circulation, is selected as the end of the analysis period, where a stationary value of the flow- and perfusion-specific component of the EIT data is again present, i.e., there is, for example, no significant global impedance change or no local significant impedance change in the region of the left heart. An impedance distribution, which corresponds, aside from an offset shift, to the impedance distribution, which was present before or at the beginning of the analysis period. will now typically become established.

The analysis period can be defined by including the heart rate. The analysis period is correspondingly shorter in case of an instantaneous or averaged heart rate increased compared to the reference state and it is correspondingly prolonged in case of a lower heart rate.

A second control signal is preferably determined and provided in another step on the basis of the two location-specific and flow- and perfusion-specific data sets. The second control signal may be used directly for visualization on a display unit, without it being necessary to additionally include provided EIT data, i.e., the data set of pixels containing impedance signals. The use of the two location-specific and flow- and perfusion-specific data sets is therefore advantageous.

The second control signal may be used for output to a display unit directly or indirectly connected to the EIT device and for transmission into a data network (LAN, WLAN, PAN, Cloud). The effort needed for data set management (addressing) and for data processing, which effort must be made for the visualization with the second control signal, is markedly reduced (by approx. 30% to 50%) compared to the provision of the data set of pixels containing impedance signals in combination with the first control signal.

In a preferred embodiment of the process/method, a separation into location-specific, flow- and perfusion-specific data sets is performed in an additional step before or after the determination of the location-specific data sets. A signal separation for determining flow- and perfusion-specific data sets is carried out, for example, as explained and described in U.S. Pat. No. 9,384,549 B2.

In a preferred embodiment of the process/method, a blood volume flow (PBF) through the lungs and/or a blood volume (PBV) within the lungs are determined and provided in an additional step after the determination of the two location-specific data sets or of the two location-specific, flow- and perfusion-specific data sets as an indicator, which indicates the state of perfusion of the lungs.

In a preferred embodiment of the process/method, a third control signal is determined and provided in an additional step on the basis of the indicator, which indicates the state of perfusion of the lungs, especially on the basis of the blood volume flow (PBF) through the lungs or on the basis of the blood volume (PBV) and on the basis of the data set of pixels.

The third control signal may be used for an output to a display unit directly or indirectly connected to the EIT device and for transmission into a data network (LAN, WLAN, PAN, Cloud).

Before the determination of the data set (CRIC) with cardiac-related impedance changes, a common data set of ventilation-specific signals is provided in a preferred embodiment of the process/method with the data set of pixels that represent the superimposition of cardiac-related signal components in regions of the lungs, of the heart or of the thorax with signal components, which represents the spread of the predefined quantity of liquid of the indicator solution in regions of the lungs, of the heart or of the thorax, and signal separation is carried out from the common data set to provide the data set of pixels with impedance signals.

The signal separation of ventilation-specific and cardiac-related signals to determine the data set (CRIC) with cardiac-related impedance changes is carried out, for example, by averaging over time over a greater number of cardiac cycles, by the high-pass or band-pass filtering in the frequency range or by means of processes that are based on the use of principal component analysis (PCA).

An application with high-pass/band-pass filterings is explained in the scientific publication of Frerichs I., Pulletz S., Elke G., Reiffenscheid F., Schadler D., Scholz J., Weiler N.: “Assessment of changes in distribution of lung perfusion by electrical impedance tomography,” Respiration, 2009: pp. 3-4, as well as Vonk Noordegraaf A., Kunst P W, Janse A., Marcus J T, Postmus P E, Faes T J, de Vries P M: “Pulmonary perfusion measured by means of electrical impedance tomography,” Physiology Measurements, 1998: pp. 265-267.

An application of the principal component analysis in connection with EIT data is described in the scientific publication by Deibele J M, Luepschen H., Leonhardt S.: Dynamic separation of pulmonary and cardiac changes in electrical impedance tomography,” Physiology Measurement, 2008: pp. 2 to 6.

A comparison of the determined data set, which indicates a relative power distribution/amplitude distribution of the cardiac-related impedance signals in a predefined frequency range, and the indicator, which indicates the state of perfusion of the lungs, is carried out by means of at least one comparison value in an additional step in a preferred embodiment. The at least one comparison value may be formed here as a single comparison value or from a combination or from combinations of comparison values from a group of comparison values. The group of comparison values has one or more of the different comparison values, especially a data set determined chronologically prior to the determined data set or an indicator of the same patient, which indicator was determined chronologically before the determined indicator, a data set determined chronologically prior to the determined data set or an indicator of another patient, which indicator was determined chronologically before the determined indicator, or a mean typical data set or a mean typical indicator of a class of patients.

A fourth control signal, which indicates a piece of information concerning the situation of the patient as a deviation of a current patient situation from a desired or normal situation, a classification of a ventilation situation, and a trend in the course of the disease, especially a progress of recovery, is determined and provided on the basis of the comparison.

The fourth control signal may be used for an output to a display unit directly or indirectly connected to the EIT device and for transmission into a data network (LAN, WLAN, PAN, Cloud).

In another preferred embodiment of the process/method, visualization is performed in an additional step on the basis of the first, second or fourth control signal with pieces of information concerning a local two-dimensional or three-dimensional position of the two location-specific data sets and/or of the location-specific, flow-specific and perfusion-specific data sets in the region of the heart, of the lungs or of the thorax in a frontal or transverse view of the lungs or of the heart.

The visualization preferably shows the location-specific and/or location-specific, flow-specific and perfusion-specific data sets in the region of the heart in a image visualization as marked regions (ROI A, ROI B), (ROI A′, ROI B′) in a transverse view of the lungs. The transverse view represents a horizontal section in the plane of the electrodes arranged on the thorax.

The indicator, which indicates the state of perfusion of the lungs and/or the blood volume flow (BPF) and/or the blood volume (PBV) or in the form of a numerical value or in the form of a curve of a time curve, is outputted on the basis of the third control signal in another preferred embodiment of the process/method.

The visualization preferably shows the blood volume and/or the blood volume flow as numerical values, in the form of diagram, for example, bar graphs, in relation to comparison values of blood volumes and/or blood volume flow, as a time curve of the blood volume and/or blood volume flow or time curve of changes of the blood volume and/or blood volume flow in graphic representation of a curve or time curve.

The embodiments described represent, each in itself as well as in combination with one another, special embodiments of the process/method according to the present invention for processing and visualizing data obtained by means of an electrical impedance tomography device (EIT) in respect to a state of perfusion of the heart and lungs. Advantages arising through the combination or combinations of a plurality of embodiments and further embodiments are covered as well, even though not all possibilities of combination of embodiments are explained in detail for this. The above-described embodiments of the process/method according to the present invention may also be configured in the form of a computer-implemented process/method as a computer program product with a computer, wherein the computer is prompted to execute the above-described process/method according to the present invention when the computer program is executed on the computer or on a processor of the computer or on a so-called “embedded system” as part of a medical device, especially of the EIT device. The computer program may also be stored on a machine-readable storage medium. In an alternative embodiment, a storage medium may be provided, which is intended for storing the above-described, computer-implemented process/method and can be read by a computer. It is within the scope of the present invention that it is not absolutely necessary to carry out all steps of the process/method on one and the same computer, but they may also be carried out on different computers, for example, in a form of the cloud computing described in more detail before. The sequence of the process/method steps may possibly also be varied. It is possible, furthermore, that individual sections of the above-described process/method may be carried out in a separate unit, which is, for example, available commercially in itself (e.g., on a data analysis system preferably arranged in the vicinity of the patient), and other parts may be carried out on another, commercially available unit (e.g., on a display and visualization unit), which is arranged, for example, as a part of a hospital information system preferably in a room set up for monitoring a plurality of hospital rooms, quasi as a distributed system.

The present invention was described above according to a first aspect of the present invention for the process/method according to the present invention for processing and visualizing data of an electrical impedance tomography device (EIT) in respect to a state of perfusion of the lungs. According to an additional first aspect of the present invention, a device for carrying out the process/method for processing and visualizing data of an electrical impedance tomography device (EIT) in respect to a state of perfusion of the lungs is provided.

According to another aspect of the present invention, a system according to the present invention for processing and visualizing data of an electrical impedance tomography device (EIT) in respect to a state of perfusion of the lungs is provided.

The advantages described for the process/method according to the present invention can be achieved in the same manner or in a similar manner with the device for carrying out the process/method according to the present invention or with the system according to the present invention as well as with the described embodiments of the device or of the system.

Furthermore, the embodiments described and their features and advantages of the process/method can be applied to the device and to the system, just like the described embodiments of the device and of the system can also be applied to the process/method.

All of the advantages that can be achieved with the described device or with the described system can be achieved in the same manner or in a similar manner with the process/method for processing and visualizing data of an electrical impedance tomography device (EIT) in respect to a state of perfusion of the lungs, especially to a blood volume flow into the lungs and to a blood volume within the lungs, which said process/method is described as the first aspect of the present invention. All the advantages of the process/method for processing and visualizing data of an electrical impedance tomography device (EIT) in respect to a state of perfusion of the lungs, especially to a blood volume flow into the lungs and to a blood volume within the lungs, may, of course, also be achieved by means of the described device and/or the described system.

The corresponding functional features of the process/method are configured by corresponding physical modules of a device, especially by hardware components (μC, DSP, MP, FPGA, ASIC, GAL), which may be implemented, for example, in the form of a processor, a plurality of processors (μC, μP, DSP) or in the form of instructions in a storage area, which are processed by the processor. The device according to the present invention for carrying out the processing and visualization of data obtained by means of an electrical impedance tomography device (EIT) for a quantifiable analysis in respect to a state of perfusion of the lungs has

a data input unit,

a control unit and

a data output unit.

The device according to the present invention is configured for receiving data by means of the data input unit. The data input unit preferably has interface elements, for example, amplifiers, A/D converters, components for overvoltage protection (ESD electrostatic discharge protection), logic elements and additional electronic components for the wired or wireless reception of the data and signals, as well as adjustment elements, such as code or protocol conversion elements for adapting the signals and data for the further processing in the control unit.

The control unit is preferably configured as a computing and control unit, e.g., in the form of a microcontroller (μC) or microprocessor (μP) with additional functions for processing the data obtained by means of an electrical impedance tomography device (EIT) for a quantifiable analysis. The control unit is configured with functions for data processing, with functions for coordinating data sets, as well as for coordinating data computing operations and function and computing sequences, which are present, for example, in the form of source code in a higher program language (C, Java, Algol, Fortran) or of a machine language (assembler) in a memory (RAM, ROM, EEPROM) or storage medium (hard drive, USB stick) associated with the control unit, which implement the present invention with processing and visualization of data of an electrical impedance tomography device (EIT) in respect to a state of perfusion of the lungs, especially a blood volume flow into the lungs and of a blood volume within the lungs. The control unit has for this purpose elements for data processing, computation and sequential control, such as microcontrollers (μC), microprocessors (μP), signal processors (DSP), logic elements (FPGA, PLD), memory components (ROM, RAM, SD-RAM) and combination variants thereof, for example, in the form of an “Embedded System.”

The data output unit is configured to generate and provide output signals and/or control signals. The output signal is preferably configured as a video signal (e.g., Video Out, Component Video, S-Video, HDMI, VGA, DVI, RGB) to make possible a graphic, numeric or image visualization of the state of perfusion of the lungs on a display unit connected to the output unit in a wireless or wired manner (WLAN, Bluetooth, WiFi) or on the output unit itself.

The carrying out of the process/method for processing and visualizing data obtained by means of an electrical impedance tomography device (EIT) in respect to a state of perfusion of the heart and lungs is implemented according to the present invention by the device for carrying out the process/method for processing and visualizing data obtained by means of an electrical impedance tomography device (EIT) in respect to a state of perfusion of the heart and lungs such that
a data set of pixels with impedance signals, which represent a superimposition of cardiac-related signal components in regions of the lungs, of the heart or of the thorax, with signal components, which represent a spread of a predefined quantity of liquid of an indicator solution in regions of the lungs, of the heart or of the thorax during a breath-hold phase, is provided by means of the data input unit on the basis of the data obtained by means of the electrical impedance tomography device (EIT) on a signal waveform located within an observation/analysis period,
a data set, which represents information concerning at least one cardiac function, especially a heart rate, is provided by means of the data input unit,
a data set containing cardiac-related impedance changes (CRIC) with pieces of information, which represent a pulsatile cardiac activity, especially a heart beat rate or a pulse beat of the heart in regions of the lungs, of the heart or of the thorax, is determined by means of the control unit on the basis of the data set of pixels and on the basis of the data set containing information concerning the at least one cardiac function, especially on the basis of the heart beat rate or of the pulse beat of the heart,
a data set, which indicates a relative distribution of a signal power or power density or a relative amplitude distribution of the cardiac-related impedance signals in a predefined frequency range, is determined by means of the control unit on the basis of the data set containing cardiac-related impedance changes (CRIC) containing pieces of information that indicate the pulsatile cardiac activity,
a data set, which indicates time or phase information of the cardiac activity in regions of the lungs, of the heart or of the thorax, is determined by means of the control unit on the basis of the data set containing cardiac-related impedance changes (CRIC) containing pieces of information that indicate the pulsatile cardiac activity, especially a heart beat rate or a pulse beat of the heart in regions of the lungs, of the heart or of the thorax,
two location-specific data sets classified according to an evaluation criterion are determined by means of the control unit on the basis of the data set that indicates the relative distribution of power or power density or the amplitude distribution of the cardiac-related impedance signals and/or on the basis of the data set containing pieces of time or phase information, which indicate the cardiac activity in regions of the lungs, of the heart or of the thorax, wherein a data set of the two location-specific data sets indicates a subset in the data set of pixels containing impedance signals, in which subset a blood volume flow is directed from the lungs to the heart, and an additional data set of the two location-specific data sets indicates a subset in the data set of pixels, in which subset a blood volume flow is directed from the heart to the lungs,
a first control signal, which indicates an indicator indicating a state of perfusion of the lungs, is determined by means of the control unit on the basis of the two location-specific data sets and on the basis of the data set of pixels containing impedance signals, and
the first control signal is provided by means of the data output unit.

According to another aspect of the present invention, a system according to the present invention for processing and visualizing data obtained by means of an electrical impedance tomography device (EIT) in respect to a state of perfusion of the heart and lungs is provided. The system according to the present invention has a control module having the features, properties and components of the above-described device according to the present invention, i.e., data input unit, control unit and data output unit, as well as a dosing module for dosing or administering an indicator solution, a ventilation module and an EIT module with an electrode array. The coordination between the EIT module and the ventilation module, which coordination is described in connection with the process/method, is made possible in practice, and the administration of the indicator solution, the breath-hold maneuver and the data acquisition are coordinated and controlled by the control unit, with start and duration over the observation/analysis period.

In the system according to the present invention with EIT module, ventilation module, dosing module, data input module and control module,
the initiation of a breath-hold maneuver at the ventilation module,
the initiation of an impedance measurement at the EIT module,
the acquisition of EIT data at the EIT module,
the determination of an indicator, which indicates a state of perfusion of the lungs, and
the determination and provision of a first control signal, which indicates the indicator, which indicates the state of perfusion of the lungs,
   are coordinated by means of the control module.

The provision of EIT data, the determination of the indicator, which indicates the state of perfusion of the lungs, as well as the determination and the provision of the first control signal are carried out according to the present invention in the system as it was described in connection with the process/method according to the present invention for processing and visualizing data obtained by means of an electrical impedance tomography device (EIT) in respect to a state of perfusion of the heart and lungs.

The system may be configured in different manners. For example, the ventilation module and the EIT module may thus be configured as an assembly unit or as a structurally combined device. For example, the ventilation module, the EIT module and the dosing module may thus be configured as an assembly unit or as a structurally combined device. For example, the control unit may thus be configured as an element of the ventilation module or of the EIT module. For example, the control module may be configured as a separate structural unit, which is connected in a data network (Cloud), for example, in a data network (LAN), to the other components, namely, the ventilation module, the EIT module and the dosing module. Additional components may also be integrated into the data network and thus provide data and pieces of information, which indicate states of the heart or of the cardiovascular system, such as pulse, blood pressure, oxygen saturation, breathing gas parameters, and which can be used by the control module for coordinating the interaction of the components, namely, the ventilation module, the EIT module and the dosing module.

The present invention will now be explained in more detail by means of the following figures and the corresponding description of the figures, without limitation to the present invention. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a flow chart for the processing of data of an EIT device for determining a state of perfusion of the heart and lungs;

FIGS. 2a is a schematic view of an additional embodiment of the flow chart according to FIG. 1;

FIGS. 2b is a schematic view of an additional embodiment of the flow chart according to FIG. 1;

FIGS. 2c is a schematic view of an additional embodiment of the flow chart according to FIG. 1;

FIGS. 2d is a schematic view of an additional embodiment of the flow chart according to FIG. 1;

FIGS. 2e is a schematic view of an additional embodiment of the flow chart according to FIG. 1;

FIG. 3 is a schematic view of an arrangement of an EIT device with an electrode array and syringe pump at a patient; and

FIG. 4 is a schematic view of a medical system with an EIT device.

### DESCRIPTION OF PREFERRED EMBODIMENTS

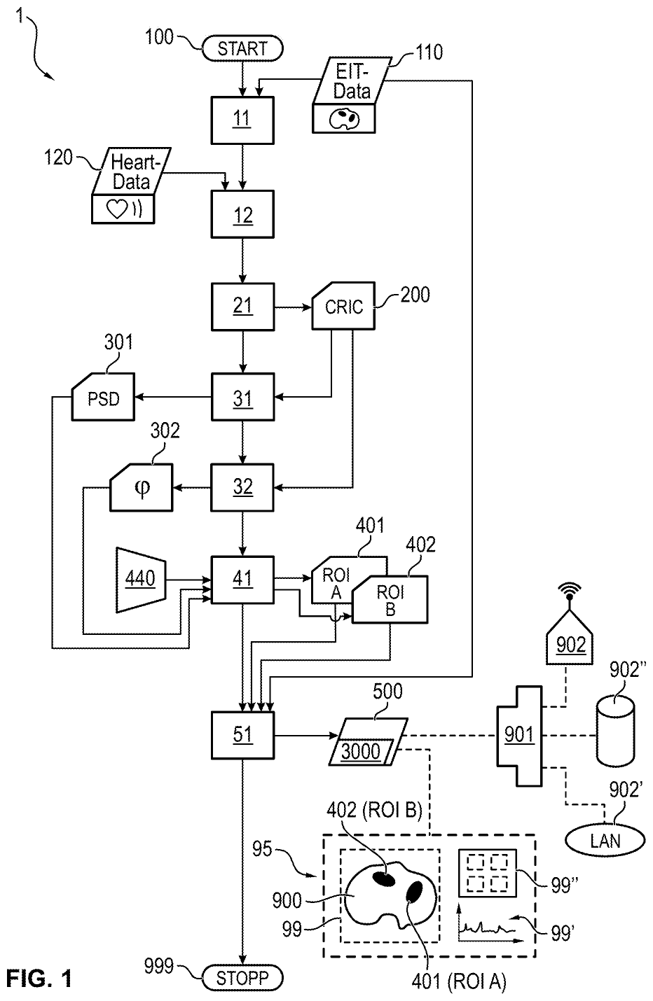

Referring to the drawings, FIG. 1 shows a flow chart, which shows the processing and the visualization of data obtained by means of an electrical impedance tomography device (EIT) in respect to the perfusion of the heart and lungs of a patient. The processing is shown on the basis of a sequence of steps 1, which begins with a start 100 and ends with a stop 999.

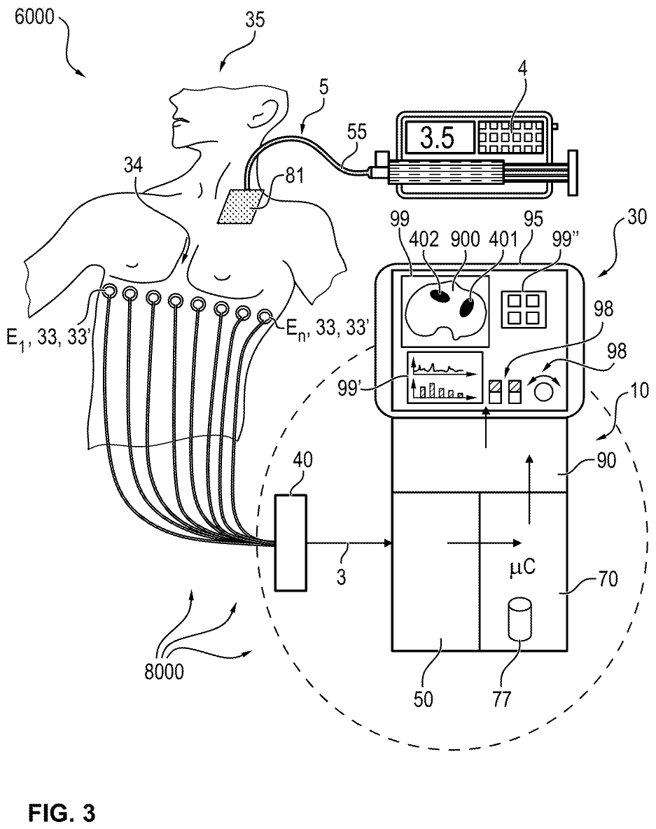

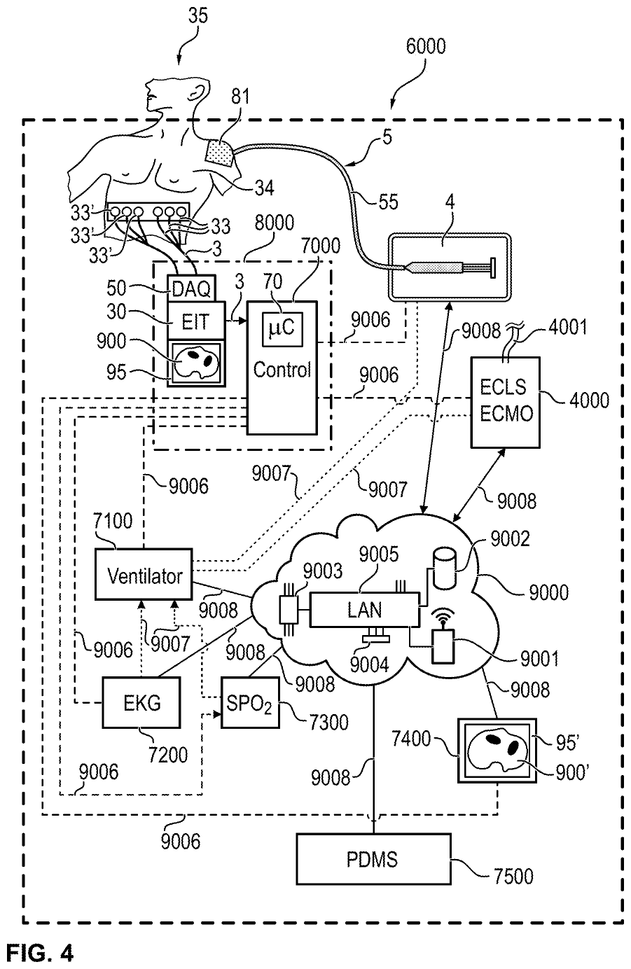

A data set of pixels 110 containing impedance signals, which contains a superimposition of cardiac-related signal components with signal components that represent the spread of a predefined quantity of liquid 55 (FIG. 3, FIG. 4) of an indicator solution, is provided in a first step 11. The data set of pixels 110 thus has data that represent a superimposition of cardiac-related signal components in regions of the lungs, of the heart or of the thorax with signal components that represent the spread of the quantity 55 (FIG. 3, FIG. 4) of the indicator solution in the regions of the lungs, of the heart or of the thorax. The spread of the quantity 55 (FIG. 3, FIG. 4) of the indicator solution results from the fact that the predefined quantity 55 of liquid of the indicator solution is injected into the bloodstream of the patient 35 (FIG. 3) during the detection of data 3 (FIG. 3) with an electrical impedance tomography device 30 (FIG. 3) on a patient 35 (FIG. 3). The injection of the quantity 55 of indicator solution by means of an invasive infusion feed 81, for example, in the form of a saline solution, may be administered via a central or peripheral venous catheter. As an alternative, administration via a lumen of a Swan-Ganz catheter is also possible. Typical access paths are blood vessels in the neck of the patient 35, for example, the internal jugular vein. If the data set of pixels 110 containing impedance signal is acquired during a period without breathing activity, be it inhalation or exhalation by the patient 35 (FIG. 3), no effects of breathing or ventilation are contained in the data set 110. In this case, the data set 110 thus contains no variation in the impedances or impedance differences, which would indicate the ventilation situation of the lungs of the patient 35 (FIG. 3). A duration without an effect of breathing or ventilation in the course of mechanical ventilation is typically brought about by means of a so-called breath-hold maneuver. The ventilation is controlled now for a predefined duration, either in a chronological relation to the inspiratory or expiratory pause of the ventilation, such that breathing gas does not flow either into the lungs of the patient or out of the lungs of the patient. The data set of pixels 110 containing impedance signals thus contains only the cardiac-related signal components as well as the signal components that are influenced by the spread of the quantity 55 (FIG. 3,

FIG. 4) of the indicator solution with the air circulation through the heart and lungs of the patient 35 (FIG. 3). The spread of the quantity 55 (FIG. 3, FIG. 4) of the indicator solution thus represents quasi a predefined maximum time frame of the observation/analysis period for the further processing of the data set of pixels 110 with impedance signals.

A data set 120, which represents information on at least one cardiac function, especially a heart rate, is provided in a step 12 following the first step 11. This data set 120 containing pieces of information concerning the cardiac function or the heart rate may have been obtained in different manners and is provided in this step 12. The information concerning at least one cardiac function can now be obtained as data information from a physiological monitor, from a monitor for monitoring the oxygen saturation (SpO₂), from a device for measuring an electrocardiogram (EKG) or also from an electrical impedance tomography device (EIT). Pieces of information concerning this at least one cardiac function may also be provided by combinations of devices, for example, a combination of a ventilator with an electrical impedance tomography device or from an electrical impedance tomography device with functions for EKG and/or SpO₂ measurement.

A data set containing cardiac-related impedance changes 200 (CRIC) is determined in a second step 21 on the basis of the data set of pixels 110 containing impedance signals and on the basis of the data set 120 containing pieces of information concerning the at least one cardiac function. The respective pulsatile activity of the heart is determined for this in each pixel of the data set 110 on pixels containing impedance signals.

A data set 301, which indicates a relative distribution of a signal power or a relative amplitude distribution of cardiac-related impedance signals in a predefined frequency range, is determined in a third step 31. The determination 31 of the data set 301 is carried out here on the basis of the data set 200 containing cardiac-related impedance changes (CRIC) with the pieces of information that indicate the pulsatile activity of the heart. The predefined frequency range is obtained here as a physiologically relevant range of frequencies, which characterize cardiac activities. Heart rates are typically in a range of about 40 beats per minute to 240 beats per minute and higher in case of a normal sinus rhythm. This corresponds to a spectral frequency range below 1 Hz up to 4 Hz. A determination 32 of a data set 302, which indicates time or phase information of the cardiac activity in regions of the lungs, of the heart or of the thorax, is carried out after the determination 31 of the data set 301 with the relative distribution of a signal power or with a relative amplitude distribution. This determination 32 of the data set containing time or phase information of the cardiac activity is carried out on the basis of the data set 200 containing cardiac-related impedance changes (CRIC), which contains pieces of information concerning the pulsatile activity of the heart. The data set 302 with the time or phase information contains information on subsets of the data set of pixels 110 with impedance signals, in which subsets inflows or outflows into or out of the lungs or outflows and inflows from and to the heart occur.

A determination of two location-specific data sets 401, 402 is carried out in a fourth step 41 on the basis of the data sets 301, 302. The data sets 301, which indicate the relative distribution of power or power density or the amplitude distribution of the cardiac-related impedance signals, and the data set 302 containing time or phase information of cardiac activities in the region of the lungs, are classified on the basis of an evaluation criterion 440. The data set 401, which indicates a subset in the set of pixels 110 with impedance signals, in which a blood volume flow is directed as a flow out of the lungs to the left heart, is obtained as a result of the classification. The data set 402, which indicates a subset in the data set of pixels 110 with impedance signal, in which a blood volume flow is directed as a flow from the right heart to the lungs, is obtained as an additional result of the classification. These two location-specific data sets 401, 402 thus describe regions of the lungs and/or heart, in which an exchange of blood between the lungs and the heart takes place. The two data sets 401, 402 are thus representative of locally definable regions, so-called “regions of interest” (ROI), which represent flows and flow directions in the exchange of blood between the heart and the lungs and can consequently be assigned to the so-called pulmonary circulation in the cardiovascular system of the lungs.

A first control signal 500, which indicates an indicator 3000 indicating a state of perfusion of the lungs, is determined and provided in a fifth step 51. The determination of the first control signal 500 is carried out here on the basis of the two location-specific data sets 401, 402 and on the basis of the data set of pixels 110 with impedance signals. The control signal 500 is suitable and intended for indicating the subsets 401, 402 in the data set of pixels 110 with impedance signals as a part of the data set of pixels 110 with impedance signals. The first control signal 500 is configured and intended to make possible a visualization on an element 99 of the display device 95 on a display device 95, which is schematically suggested with broken lines in this figure as an optional component. Additional optional components are shown in this FIG. 1. Thus, an element for visualizing a curve 99′ as well as an element for visualizing a numerical value 99″ are also shown as additional elements 99′ and 99″, respectively. The first control signal 500 is optionally sent to and/or provided for additional components in this FIG. 1. These optional components 901, 902, 902′, 902″ are connected with broken line in the drawings to the first control signal 500 by means of an interface 901. Network components (LAN) 902′, network or data servers 902″ as well as means for wireless data transmission 902 can be supplied with the first control signal by means of the interface 901. Provision of the data sets 401, 402 into a data network or network system is made possible in this manner in order to make it possible to display the pieces of information obtained by means of this data processing in respect to the state of perfusion and the flow conditions in the lungs and heart not only directly at the electrical impedance tomography device (EIT) 30 (FIG. 3), i.e., at the site at which the data are obtained, but to also make possible a transmission of the data to additional units in the data network, for example, in a hospital network.

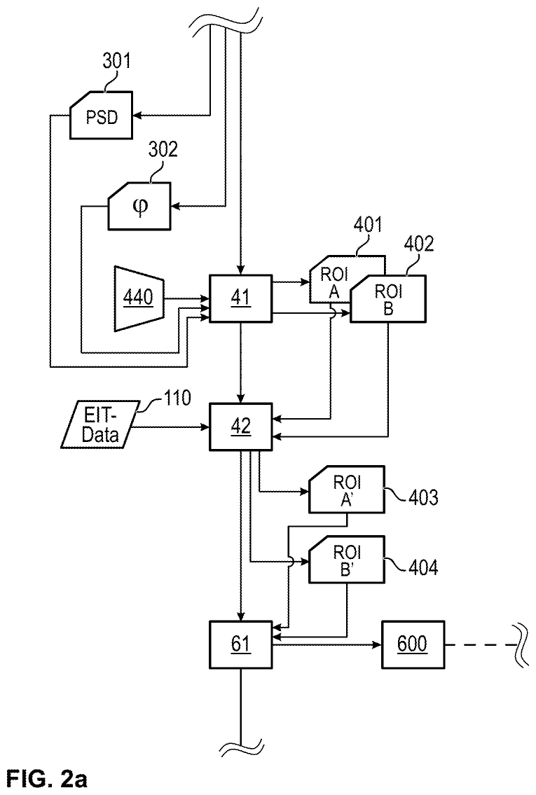

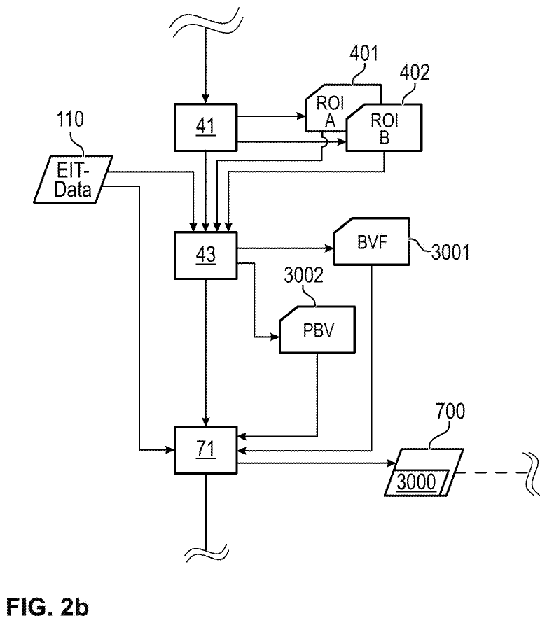

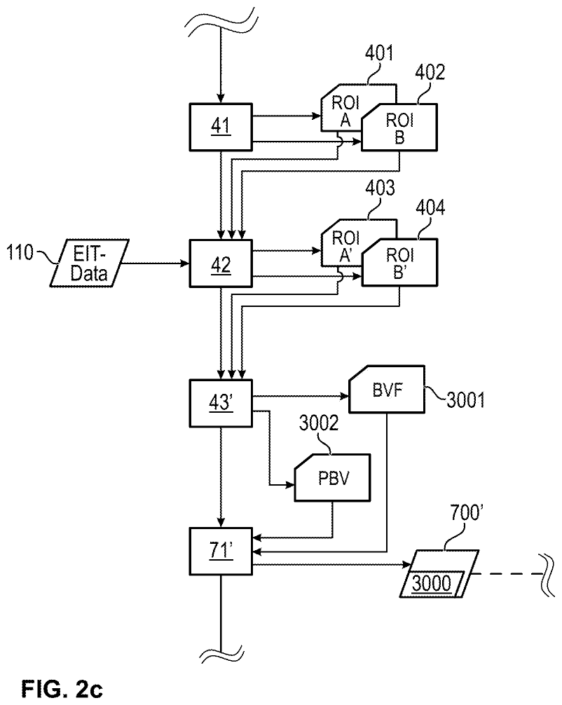

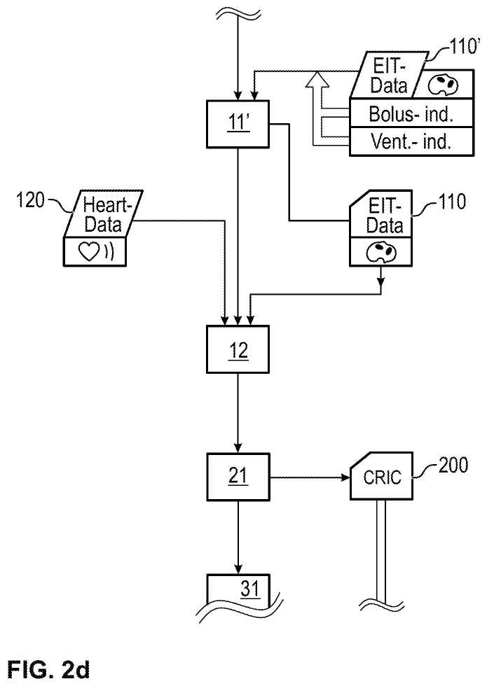

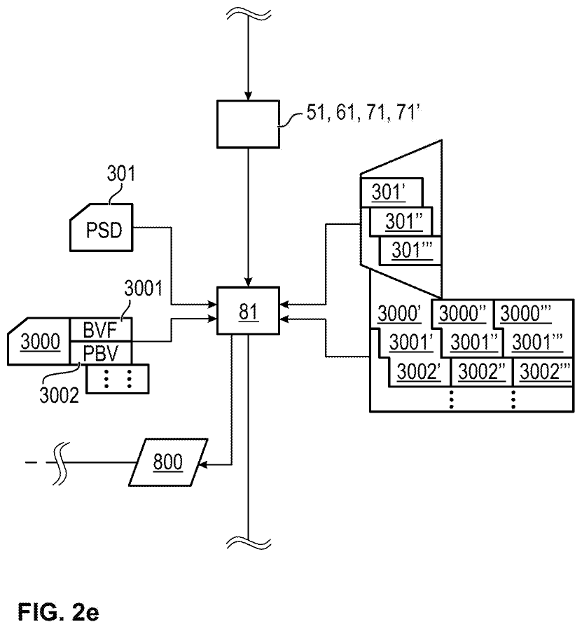

FIGS. 2a through 2e show embodiments of the sequence 1 according to FIG. 1. These embodiments have additional steps, which may additionally or alternatively be integrated in the sequence 1 or additionally in parts. Identical elements in FIGS. 1 and 2a are designated by the same reference numbers in FIGS. 1 and 2a.

It is described in FIG. 2a that a signal separation is carried out on the basis of the data set of pixels 110 containing impedance signals and/or on the basis of the two location-specific data sets 401, 402 before or after the determination 41 of the location-specific data sets 401, 402. Two location-specific and flow- and perfusion-specific data sets 403, 404 are determined and provided as a result. Determination and provision of a second control signal 600 is carried out following this signal separation 42 in a further step 61 on the basis of the two location-specific and flow- and perfusion-specific data sets 403, 404.

These additional steps 42, 61 described in FIG. 2a are integrated into the sequence 1 according to FIG. 1, as is graphically suggested in FIG. 2a, after the fourth step 41 with the determination of the location-specific data sets 401, 402. Compared with FIG. 1, this determination of the two location-specific and flow- and perfusion-specific data sets 403, 404 has the advantage that the second control signal 600 can be used directly for the visualization 900, 900′ (FIG. 1, FIG. 3, FIG. 4), without an inclusion of the data set of pixels 110 containing impedance signals being necessary for the output.

FIG. 2b shows an expansion of the sequence 1 according to FIG. 1 and likewise of the partial sequence in FIG. 2a. Identical elements in FIGS. 1, 2a as well as 2b are designated by the same reference numbers in FIGS. 1, 2a as well as 2b. The indicator 3000 determined for the state of perfusion of the lungs in sequence 1 of FIG. 1 is determined specifically in a step 43 after the determination 41 of the location-specific data sets 401, 402. Including the data set of pixels 110 with impedance signals, a blood volume flow PVF 3001 through the lungs is determined in step 43. Based on the blood volume flow PVF 3001, a blood volume (PBV) 3002 within the lungs is additionally determined in a further step 43 on the basis of the location-specific data sets 401, 402 and of the data set of pixels 110 containing impedance signals. The indicator 3000, which indicates the state of perfusion of the lungs, can thus be configured in the form of the blood volume flow PVF 3000, as well as also of the blood volume (PBV) 3002 and determined and provided in a further step 71 as a third control signal 700 on the basis of the indicator 3000, PVF 3001 as well as (PBV) 3002. These additional steps in FIG. 2b are integrated into the sequence 1 according to FIG. 1, and the provision 71 of the control signal 700 is suitable for a visualization 900, 900′ (FIG. 1, FIG. 3, FIG. 4).

FIG. 2c shows an alternative embodiment of FIG. 2b. Identical elements in FIGS. 1, 2a, 2b, 2c are designated by the same reference numbers in FIGS. 1, 2a, 2b, 2c. Unlike in FIG. 2b, the two location-specific and flow- and perfusion-specific data sets 403, 404 are used in FIG. 2c in the determination 43′ instead of the location-specific data sets 401, 402 to determine the blood volume flow PVF 3001 through the lungs or the blood volume (PBV) 3002 as the indicator 3000, which indicates the state of perfusion of the lungs. An alternative third control signal 700′ is determined and provided in an additional step 71′ on the basis of the indicator 3000, which indicates the state of perfusion of the lungs. The integration of the steps according to FIG. 2c is carried out in a comparable manner as is described in connection with FIG. 2b, with a possibility of connection to a visualization 900, 900′ (FIG. 1, FIG. 3, FIG. 4).

An alternative embodiment of the data provision 11 (FIG. 1) of sequence 1 according to FIG. 1 is shown in FIG. 2d. Identical elements in FIG. 1 and in FIG. 2d are designated by the same reference numbers in FIG. 1 and FIG. 2d. Instead of a data set of pixels 110 containing impedance signals, which represent a superimposition of cardiac-related signal components in the lungs with signal components that represent the spread of the quantity 55 (FIG. 3, FIG. 4) of the indicator solution in regions of the lungs and of the thorax, a common data set of pixels 110′ containing impedance signals is provided, which contains ventilation-specific signal components 130, which are based on effects of inhalation/exhalation in the lungs due to breathing or ventilation, in addition to the cardiac-related signal components and to the signal components due to the spread of the quantity 55 (FIG. 3, FIG. 4) of the indicator solution. This data set of pixels 110′ is subjected for this to a signal separation in an additional signal processing 11′ in a further additional step. This signal separation 11′ is used to remove the ventilation-specific signals 130 from the data set 110′. A data set of pixels 110, which represents the superimposition of cardiac-related signal components in regions of the lungs and of the heart or of the thorax with signal components that represent the spread of the predefined quantity of liquid 55 (FIG. 3, FIG. 4) of the indicator solution in regions of the lungs, of the heart or the thorax, is obtained, in turn, as a result of the signal separation 11′. The integration of step 11′ is carried out according to this FIG. 2d in sequence 1 of FIG. 1 as an additional step 11′ or as part of the first step shown and described in FIG. 1 in sequence 1 with provision 11 of the data set of pixels 110 containing impedance signals.

FIG. 2e shows an additional, further processing of the signals and results of sequence 1 of FIG. 1, as well as additional embodiments according to FIGS. 2b as well as 2c. Identical elements in FIGS. 1, 2b, 2c, 2e are designated by the same reference numbers in FIGS. 1, 2b, 2c, 2e. The data sets determined in FIGS. 1, 2b, 2c, which indicates a relative power distribution/amplitude distribution of the cardiac-related impedance signals in a predefined frequency range, as well as the indicator 3000, which indicator indicates the state of perfusion of the lungs, are compared in a further step 81 with comparison values 301′, 301″, 301′″ of the relative power/amplitude distribution and/or also of the indicator 3000′, 3000″, 3000′″, 3001′, 3001″, 3001′, 3002′, 3002″, 3002′″. The indices ′, ″, ′″ indicate different situations, in which the comparison values have been determined. The index ′ designates a data set 301′ as an indicator 3000′, 3001′, 3002′ of the same patient. The index ″ designates a data set 301″ as an indicator 3000″, 3001″, 3002″ of another patient. The index ′″ designates a typical data set 301′″ as an indicator 3000′″, 3001′, 3002′″ of a class of patients. As a result of this comparison 81, a fourth control signal 800 is generated, which is provided for an output, for example, a visualization 900, 900′ (FIG. 1, FIG. 3, FIG. 4) and can thus be used in connection with the sequence 1 of FIG. 1.

FIG. 3 shows a schematic view of an arrangement of an EIT system 8000 with an EIT device 30 and electrode array 33 with a plurality of electrodes E₁, . . . Eₙ in combination with a syringe pump 4 in a common embodiment as a medical system 6000. The medical system 6000 according to this FIG. 3 makes possible a common functionality for carrying out the process/method of visualization of data obtained by means of an electrical impedance tomography device (EIT) in respect to the perfusion of the heart and lungs of a patient according to sequence 1 according to FIG. 1. Identical elements in FIGS. 1, 2a, 2b, 2c, 2d, 2e, 3 are designated by the same reference numbers in FIGS. 1, 2a, 2b, 2c, 2d, 2e, 3. The electrode array 33 with the electrodes E₁, . . . Eₙ 33′ is arranged on the upper body (thorax) of a patient 35. A measured data acquisition and feed unit 40 is configured to feed a signal, preferably an alternating current (current feed) or also an alternating voltage (voltage feed) at a respective pair of electrodes 33′ in a measurement cycle. The voltage signals resulting from the alternating current feed (current feed) are acquired as signals at the other electrodes 33′ by the measured value acquisition and feed unit 40 and are provided as EIT data 3 to the data input unit 50. In addition to the measured data acquisition, the syringe pump 4 is likewise arranged at the patient 35 via an infusion line 5 and a site for invasive infusion feed 81, configured, for example, as an access in the cervical region of the patient 35. The EIT data 3 provided are fed in the EIT device 30 to a control unit 70 via a data input unit 50. A memory 77, which is configured to store a program code, is provided in the control unit 70. The run of the program code is coordinated by a microcontroller arranged as an essential element in the control unit or by another embodiment of computing elements (FPGA, ASIC, μP, μC, GAL). The computing and control unit 70 is thus prepared and intended to coordinate the sequence of steps shown in FIGS. 1, 2a, 2b, 2c, 2d, 2e and to carry out the steps shown with comparison operations, computation operations, storage and data organization of the data sets, for example, of the data sets 200, 301, 302 (FIG. 1), 401, 402 (FIG. 2a), 403, 404 (FIG. 2b). The values determined by the control unit 70 are provided by means of a data output unit 90 as control signals 500 (FIG. 1), 600 (FIG. 2a), 700 (FIG. 2b), 700′ (FIG. 2c), 800 (FIG. 2e) and results 3000 (FIG. 1), 3001, 3002 (FIGS. 2b, 2c) to a data output unit 90 and are visualized 900 on a display device 95. An alternative of a visualization 900′ (FIG. 4) on an external display device 95′ (FIG. 4) is shown in the embodiment of the medical system 6000 shown in FIG. 4. In addition to the visualization 900, additional elements 99′, for example, operating elements 98, elements 99′ for displaying numerical values or elements 99′ for displaying time curves or curves, are also present on the display device 95.

The syringe pump interacts with the EIT device 30 as follows: A predefined quantity 55 (bolus) of an indicator solution is injected by the syringe pump 4 into the blood circulation of the patient 35 via the infusion line 5 and the site of the invasive infusion feed 81. This quantity 55 of indicator solution flows through the blood circulation of the patient 35 with the blood flow and then reaches the right atrium of the heart of the patient 35 with the oxygen-depleted blood having a high level of carbon dioxide. This quantity 55 of the indicator solution then enters from there the lungs of the patient 35 with the blood flow and then again the blood circulation back from the lungs with the blood having a high oxygen level and having been freed from carbon dioxide for supplying organs and muscles of the patient 35 with oxygen. The flow of the quantity 55 of the indicator solution through the lungs brings about a change in the conductivity as a measurement effect, which can be detected by means of the EIT device 30 and the associated electrode array 33 as a locally and chronologically significant change in the impedances in both a region 402 in the plane of the electrode array 33, into which region the quantity 55 of the indicator solution flows through the plane of the electrode array 33 with the blood flow from the heart into the lungs, and also in a region 401 in the plane of the electrode array 33, in which region the quantity 55 of the indicator solution flows back into the heart from the lungs with the blood flow from the lungs into the heart through the plane of the electrode array 33. The procedure described in FIG. 1 with the sequence 1 makes it possible, when it is carried out by the control unit 70, to determine these two regions (ROI, Regions of Interest) 401, 402 in the image visualization or visualization 900 of the EIT data 3, in which the quantity 55 of the indicator solution flows from the lungs into the heart (ROI A) and flows again out of the heart to the lungs (ROI B). These regions (ROI A, ROI B) correspond to the regions that are designated as location-specific data sets 402, 401 in FIG. 1 in sequence 1.

FIG. 4 shows a schematic visualization of a medical system 6000 with an EIT device. Identical elements in FIGS. 1, 2a, 2b, 2c, 2d, 2e, 3 and 4 are designated by the same references in FIGS. 1, 2a, 2b, 2c, 2d, 2e, 3, 4.

The medical system 6000 has as additional components, in addition to the components according to FIG. 3 with an EIT system 8000 with an EIT device 30 and infusion pump 4, a ventilator 7100, an EKG measuring device 7200, an SpO₂ measuring device 7300, a visualizing device 7400, a patient management system 7500, an extracorporeal lung assist device 4000 in a data interaction among and with one another in a data network system 9000 (cloud).

The data network system 900 has telemetry components (WLAN, Bluetooth) 9001, memory (file server, hard drive memory, hard disk), central and decentralized computers (servers) 9002, switching and coordination units (router, switch) 9003, units 9004 (HUB) for level adjustment and level amplification. The devices 7100, 7200, 7300, 4000, 7400, 8000, 7500 and the components 9001, 9002, 9003, 9004 are connected in a medical system 6000 into a network 9005 in the data network system 9000 via data connections 9008. These data connections 9008 in the data network system 9000 are indicated by solid lines in this FIG. 4.

The EIT system 8000 is configured as is described in connection with FIG. 3. The EIT system 8000 thus comprises an EIT device 30 with control unit 70, data input unit 50, electrode array 33 with a number of electrodes E₁, . . . Eₙ 33′ and an output unit 95 suitable for visualization 9000. A measured value acquisition unit 40 (FIG. 3) is arranged at or in the data input unit 50 for signal feed and signal acquisition as well as for preprocessing (amplification, filtering) of the signals of the electrodes 33′. A data output unit 90 (FIG. 3) is arranged at or in the display device 95. The acquired signals enter the control unit 70 in the EIT device 30 via the data input unit 50 as EIT data 3 from the electrode array 33 with the plurality of electrodes 33′. The EIT system 8000 with the components 30, 40, 50, 70, 90, 95 is shown in this FIG. 4 with an outer border in the form of a dash-dotted line.

Additional data connections 9006, 9007 are shown in the medical system 6000 in this FIG. 4. Thus, there are direct data connections 9006 in the medical system 6000 from the devices 7100, 7200, 7300, 4000, 7400 to the EIT system 8000 or the EIT device 30. These direct data connections 9006 to the EIT device 30 are indicated by broken lines in this FIG. 4. In addition, there are direct data connections 9007 from different components 7200, 7300, 4000 to the ventilator 7100 in the medical system 6000. These direct data connections 9007 to the ventilator 7100 are indicated by broken lines in this FIG. 4. Direct interactions between the EIT system 8000 and/or the ventilator 7100 and the other devices 7200, 7300, 4000 are possible via the direct data connections 9006, 9007 without the inclusion of the data network system 9000.

All components of the medical system 6000 can be caused to interact and coordinate with one another with the inclusion of the data network system 9000 via the data connections 9008. This coordination is preferably carried out by a central control unit 7000. This central control unit 7000 is shown in this FIG. 4 as a component of the EIT system 8000 connected to the EIT device or also as a part of the EIT device 30. The central control unit 7000 can coordinate the functionality described in connection with FIG. 3 with processing and visualization of EIT data 3 in respect to a state of perfusion of the heart and lungs. This coordination may take place such that the infusion pump 4 as well as the ventilator 7100 and additional components are coordinated by the central control unit 7000 such that the administration of the quantity 55 of indicator solution takes place with the control of the ventilator 7100 preferably during a special maneuver of the mechanical ventilation in a so-called breath-hold phase and EIT data 3 are at the same time acquired by means of the EIT device 30 over the period of the breath-hold phase as an observation/analysis period. The electrodes 33′ of the electrode array 33 are arranged on the thorax 34 of a patient 35. The syringe pump 4 brings about the administration of the quantity 55 of the indicator solution via an infusion line 5 at a site of an invasive infusion feed 81, in agreement in this FIG. 4 with the view shown in FIG. 3 in the region of the neck or shoulder of the patient 35. The extracorporeal lung assist device 4000 is connected to the patient 35 by means of a blood circulation connection 4001, which is only suggested in this FIG. 4, in order to feed oxygen to the patient and to remove carbon dioxide from the blood circulation of the patient 35. The blood circulation connection 4002 of the extracorporeal lung assist device (ECLS, ECMO) 4000 is used to connect the blood circulation of the patient 35 to the extracorporeal lung assist device 4000, for example, through an invasive access (arterial/venous) in the region of the groin of the patient 35 or at other suitable locations on the body.

A possibility of interaction of the ventilator 7100 with the extracorporeal lung assist device 4000 can be configured such that the ventilator 7100 coordinates, on the one hand, the time at which the quantity 55 of indicator solution is administered with the configuration of the ventilation and ventilation maneuver (breath-hold phase) and also takes on the task of supplying the patient with oxygen from the ventilator 7100 during the duration of the breath-hold phase.

The network 9005 in the data network system 9000 is configured to exchange the data or instructions between the individual components 4000, 7100, 7200, 7300, 7400, 7500, 8000 physically (wired line connections, optical data connections, telemetric data connections) and in terms of data technology (transmission protocols, error management), and to organize the corresponding infrastructure with the components 9001, 9002, 9003, 9004 and data connection 9008. The data connections 9008 may take place both in a wireless manner telemetrically or in a wireless manner optically. The visualization device 7400 is present in the system 6000 as an additional or alternative display device 95′ to the display device 95 present in the EIT device 30 and/or in the EIT system 8000. This alternative or additional display device 95′ may be arranged, for example, in a monitoring room, in which the clinical staff receives a display of a large number of information and/or data of individual patients or of a plurality of patients and can thus use this information in respect to an assessments of health situations of individual patients and for a comprehensive monitoring of these patients.

The central control unit 7000 is shown in this FIG. 4 as an embodiment of the control unit 70 of the EIT system 8000 with the EIT device 30 of the display device 95 and with the data input device 50.

However, other embodiments with arrangement of the central control unit 7000 in the system 6000 with possibilities of data provision 11 (FIG. 1, FIG. 2d), possibilities of data processing 21, 31, 41, 51 (FIG. 1), 42, 61 (FIG. 2a), 43, 71 (FIG. 2b), 43′, 71′ (FIG. 2c), 81 (FIG. 2e) in the EIT system 8000, data network system 9000 or medical system 6000 are also covered in the sense of the present invention. Thus, the central control unit 7000 may also preferably be configured, for example, as a part of the ventilator 7100 or as a part of the data network system 9000, for example, on a suitable data server 9002 arranged specially for this purpose in this data network system 9000. This leads to the possibility that the processing for a visualization of EIT data 3 in respect to a state of perfusion of the heart and lungs, as well as the coordination of the syringe pump 4 and of the ventilator 7100 in respect to the administration of the quantity 55 of indicator solution and for the organization of special ventilation settings and breath-hold phases at the ventilator 7100 can be carried out from a location located outside the EIT system 8000, quasi by as switching station or remote assistance station. In addition, the integration of the visualization into the data network system 9000 offers the possibility of using special computing rules detached from the EIT device 30 or EIT system 8000 independently from the site of the measurement and to use them for the use in the assessment of the situation of the patient 35.

These are some of the advantages that arise in connection with the components 400, 7100, 7200, 7300, 7400, 7500, 8000, especially for the EIT system 8000 as a part in a data network system 9000, but they are described in this FIG. 4 only as an example and without a claim to completeness.

While specific embodiments of the invention have been shown and described in detail to illustrate the application of the principles of the invention, it will be understood that the invention may be embodied otherwise without departing from such principles.

1. A process for processing and visualizing electrical impedance tomography device data (EIT data) obtained by means of an electrical impedance tomography device in respect to a perfusion of the heart and lungs of a patient the process comprising the steps of
providing a data set of pixels containing impedance signals, which represent a superimposition of cardiac-related signal components in regions of the lungs, in regions of the heart or in regions of the thorax with signal components, which represent the spread of a predefined quantity of fluid of an indicator solution in regions of the lungs, in regions of the heart or in regions of the thorax during a breath-hold phase, on the basis of the data obtained by means of the electrical impedance tomography device via a signal waveform located within an analysis period,
providing a data set, which represents information concerning at least one cardiac function,
determining a data set with cardiac-related impedance changes containing information that represents a pulse beat of the heart in regions of the lungs, in regions of the heart or in regions of the thorax on the basis of the data set of pixels and on the basis of the data set containing information concerning the at least one cardiac function,
determining a data set, which indicates a relative distribution of a signal power or power density or a relative amplitude distribution of the cardiac-related impedance signals in a predefined frequency range, on the basis of the data set containing cardiac-related impedance changes with information that indicates the pulsatile activity of the heart,
determining a data set, which indicates time or phase information of the cardiac activity in regions of the lungs, of the heart or of the thorax, on the basis of the data set containing cardiac-related impedance changes with information that indicates the pulsatile activity of the heart, in regions of the lungs, in regions of the heart or in regions of the thorax,
determining two location-specific data sets classified according to an evaluation criterion on the basis of the data set that indicates the relative distribution of power or powder density or the amplitude distribution of the cardiac-related impedance signals and/or on the basis of the data set containing time or phase information, which indicates the cardiac activity in regions of the lungs, in regions of the heart or in regions of the thorax, wherein a data set of the two location-specific data sets indicates a subset in the data set of pixels with impedance signals, in which subset a blood volume flow is directed from the lungs to the heart and wherein an additional data set of the two location-specific data sets indicates a subset in the data set of pixels with impedance signals, in which a blood volume flow is directed from the heart to the lungs,
determining and providing an indicator, which indicates a state of perfusion of the lungs on the basis of the two location-specific data sets and on the basis of the data set of pixels with impedance signals, and
determining and providing a first control signal, which indicates the indicator indicating the state of perfusion of the lungs.

2. A process in accordance with claim 1, wherein a signal separation is carried out, in an additional step before or after the determination of the location-specific data sets, on the basis of the data set of pixels with impedance signals and/or on the basis of the two location-specific data sets, and two location-specific, flow-specific and perfusion-specific data sets are provided.

3. A process in accordance with claim 2, wherein a second control signal is determined and provided in an additional step on the basis of the two location-specific and flow- and perfusion-specific data sets.

4. A process in accordance with claim 1, wherein a blood volume flow through the lungs or a blood volume within the lungs is determined and provided in an additional step, after the determination of the data sets, as an indicator, which indicates the state of perfusion of the lungs, on the basis of the two location-specific data sets and of the data set of pixels with impedance signals.

5. A process in accordance with claim 1, wherein a blood volume flow through the lungs or a blood volume within the lungs is determined and provided in an additional step after the determination of the data sets as an indicator, which indicates the state of perfusion of the lungs, on the basis of the location-specific and flow- and perfusion-specific data sets.

6. A process in accordance with claim 4, wherein an additional control signal is determined and provided in an additional step on the basis of the indicator, which indicates the state of perfusion of the lungs, on the basis of the blood volume flow through the lungs or on the basis of the blood volume and on the basis of the data set of pixels.

7. A process in accordance with claim 4, wherein an additional control signal is determined and provided in an additional step on the basis of the indicator, which indicates the state of perfusion of the lungs, on the basis of the blood volume flow through the lungs or on the basis of the blood volume which indicates the state of perfusion of the lungs.

8. A process in accordance with claim 1, wherein before the determination of the data set with cardiac-related impedance changes, a common data set of ventilation-specific signals is provided with the data set of pixels, which represent the superimposition of cardiac-related signal components in regions of the lungs, of the heart or of the thorax with signal components, which represent the spread of the predefined quantity of liquid of an indicator solution, with ventilation-specific signals, and a signal separation is carried out from the common data set to provide the data set of pixels containing impedance signals.

9. A process in accordance with claim 1, wherein a comparison of the determined data set, which indicates a relative power distribution/amplitude of the cardiac-related impedance signals in a predefined frequency range, and the indicator, which indicates the state of perfusion of the lungs, is carried out in an additional step by means of at least one comparison value, wherein the at least one comparison value is formed as a single comparison value or from a combination or combinations of comparison values from a group of comparison values, wherein the group of comparison values has one or more of the following comparison values.
a data set of the same patient, which was determined chronologically before the determined data set,
an indicator of the same patient, which was determined chronologically before the determined indicator,
a data set of another patient, which was determined chronologically before the determined data set,
an indicator of another patient, which was determined chronologically before the determined indicator,
a mean typical data set of a class of patients, and
a mean typical indicator of a class of patients,
wherein further control signal, which indicates information concerning the situation of the patient as
a deviation of a current patient situation from a desired or normal situation,
a classification of a ventilation situation, and
a trend in the course of the disease, is determined and provided on the basis of the comparison.

10. A process in accordance with claim 1, wherein a visualization is carried out on the basis of the control signal with pieces of information concerning a local two-dimensional or three-dimensional position of the two location-specific data sets in the region of the heart, in the region of the lungs or in the region of the thorax in a front view or transverse view of the lungs or of the heart.

11. A process in accordance with claim 6, wherein the indicator, which indicates the state ofperfusion of the lungs and/or the blood volume flow and/or the blood volume, is outputted on the basis of the additional control signal in the form of numerical values, diagrams, in relation to comparison values or of a curve of a time curve.

12. A device for carrying out a process for processing and visualizing electrical impedance tomography data obtained by means of an electrical impedance tomography device in respect to the perfusion of the heart and lungs of a patient, the device comprising
data input unit providing )a data set of pixels with impedance signals, which represent a superimposition of cardiac-related signal components, which represent the spread of a predefined quantity of liquid of an indicator solution in regions of the lungs, in regions of the heart or in regions of the thorax during a breath-hold phase, on the basis of the electrical impedance tomography data obtained by means of the electrical impedance tomography device (EIT) via a signal waveform located within an analysis period, and providing a data set, which represents information concerning at least one cardiac function, and
a control module configured for:
determining a data set with cardiac-related impedance changes with information that indicates a pulsatile activity of the heart, in regions of the lungs, in regions of the heart or in regions of the thorax, on the basis of the data set of pixels and on the basis of the data set containing information concerning the at least one cardiac function,
determining a data set, which indicates a relative distribution of a signal power or power density or a relative amplitude distribution of the cardiac-related impedance signals in a predefined frequency range, on the basis of the data set with cardiac-related impedance changes with information that indicates the pulsatile activity of the heart,
determining a data set, which indicates time or phase information of the cardiac activity in regions of the lungs, in regions of the heart or in regions of the thorax, on the basis of the data set with cardiac-related impedance changes with information that indicates the pulsatile activity of the heart,
determining two location-specific data sets classified according to an evaluation criterion on the basis of the data set that indicates the relative distribution of power or power density or the amplitude distribution of the cardiac-related impedance signals and/or on the basis of the data set with time or phase information that indicates the cardiac activity in regions of the lungs, in regions of the heart or in regions of the thorax, wherein one data set of the two location-specific data sets indicates a subset in the data set of pixels with impedance signals, in which subset a blood volume flow is directed from the lungs to the heart, and wherein an additional data set of the two location-specific data sets indicates a subset in the data set of pixels with impedance signals, in which subset a blood volume flow is directed from the heart to the lungs, and
determining and providing a control signal, which indicates an indicator which indicates a state of perfusion of the lungs on the basis of the two location-specific data sets and on the basis of the data set of pixels with impedance signals, and
a data output unit configured for determining the first control signal, which indicates the indicator indicating the state of perfusion of the lungs, by means of the data output unit.

13. A system comprising an EIT module, a ventilation module, a dosing module, a data input module and a control module configured to
initiate and coordinate a breath-hold maneuver at the ventilation module,
initiate and coordinate an impedance measurement at the EIT module,
coordinate a data acquisition of EIT data at the EIT module,
determine an indicator, which indicates a state ofperfusion of the lungs, and
determine and provide a first control signal, which indicates the indicator, which indicates the state of perfusion of the lungs.
